(19) United States
(12) Patent Application Publication
(10) Pub. No.: US 2024/0041989 A1
(43) Pub. Date: Feb. 8, 2024

(54) STRUCTURE-BASED DESIGN AND DISCOVERY OF LONG-ACTING COCAINE HYDROLASE MUTANTS WITH IMPROVED BINDING AFFINITY TO NEONATAL FC RECEPTOR FOR TREATMENT OF SUBSTANCE USE DISORDERS AND ORGANOPHOSPHORUS TOXICITY

(71) Applicant: University of Kentucky Research Foundation, Lexington, KY (US)

(72) Inventors: Chang-Guo Zhan, Lexington, KY (US); Fang Zheng, Lexington (US)

(21) Appl. No.: 18/268,575

(22) PCT Filed: Dec. 21, 2021
(86) PCT No.: PCT/US21/64647

§ 371 Date: Jun. 20, 2023

Related U.S. Application Data
(60) Provisional application No. 63/128,774, filed on Dec. 21, 2020.

(51) Int. Cl. A61K 38/46; C12N 9/18; A61P 25/30

(52) U.S. Cl. CPC A61K 38/465; C12N 9/18; C12Y 301/01008; A61P 25/30

(57) ABSTRACT

The presently disclosed subject matter describes methods of treating cocaine use disorder and organophosphorus toxicity in which a butyrylcholinesterase fusion protein is administered to a subject in need thereof.

### RELATED APPLICATIONS

This application claims the benefit of U.S. Provisional Application Ser. No. 63/128,774, filed Dec. 21, 2021, the entire disclosure of which is incorporated herein by this reference.

### GOVERNMENT INTEREST

This invention was made with government support under Grant Numbers UH2 DA041115, UH3 DA041115, and U01 DA051079 awarded by the National Institute of Health (NIH). The government has certain rights in the invention.

### SEQUENCE LISTING

This application contains a sequence listing submitted in accordance with 37 C.F.R. 1.821, entitled “13177N 2517 ZHAN Sequence Listing.txt”, created on Dec. 13, 2021, having a size of 146,609 bytes, which is incorporated herein by this reference.

### TECHNICAL FIELD

The presently disclosed subject matter relates to methods of treating cocaine use disorder and organophosphorus toxicity. More particularly, the presently disclosed subject matter relates to methods of treating cocaine use disorder and organophosphorus poisoning in which a butyrylcholinesterase fusion protein is administered to a subject in need thereof.

### BACKGROUND

Cocaine is known as one of the highly abused drugs.¹⁻³ Traditional strategies for therapeutic treatment of cocaine abuse aim to antagonize the action of cocaine on a brain target, such as dopamine transporter (DAT).⁴ Unfortunately, it would be extremely difficult to directly block the action of cocaine without interrupting the normal function of DAT, as the cocaine-binding site overlaps with the dopamine (DA)-binding site.⁵⁻⁷ There is still no FDA-approved therapeutic specific for cocaine dependence or overdose. To meet the unmet need has led to exploring biologics such as monoclonal antibodies capable of stoichiometrically binding with cocaine, vaccines that actively elicit such antibodies, and enzymes.⁸⁻¹³ A cocaine-metabolizing enzyme is expected to require a lower effective dose than antibodies, because each enzyme molecule can degrade multiple cocaine molecules, depending on the catalytic rate constant (k_{cat}) and Michaelis-Menten constant (K_{M}) of the enzyme against cocaine.

The primary endogenous cocaine-metabolizing enzyme in plasma is butyrylcholinesterase (BChE) (SEQ ID NO: 1), and the products of BChE-catalyzed hydrolysis of cocaine are biologically inactive.¹⁴⁻¹⁵ The catalytic efficiency (k_{cat}/K_{M}) of wild-type BChE against naturally occurring (−)-cocaine is, however, too low (k_{cat}=4.1 min⁻¹ and K_{M}=4.5 μM)¹⁶ to be effective. Through previous structure-and-mechanism-based computational design, human BChE mutants with considerably improved catalytic efficiency against cocaine have been discovered.^{9, 17-23} The first computationally designed cocaine hydrolase (CocH), denoted as CocH3 (i.e. the A199S/S287G/A328W/Y332G mutant),^{17, 24-26} was truncated after amino acid #529 and fused with human serum albumin (HSA)²⁷ to prolong the biological half-life for clinical development to treat cocaine dependence. This fusion protein is also known as Albu-CocH1, Albu-CocH, TV-1380, or Albu-BChE in literature.²⁷⁻³² Multiple Phase I clinical trials²⁸⁻²⁹ favorably confirmed the safety of TV-1380 for its use in humans. Subsequently, a Phase II clinical trial³¹ revealed that TV-1380 at a once-weekly dosing schedule (up to 300 mg per dose) significantly and dose-dependently decreased cocaine intake in the treatment groups compared to the placebo group, but its biological half-life (t_{1/2}=8 hours in rats and 43-77 hours in humans) was not long enough for the practical cocaine dependence treatment to keep abstinence from cocaine using TV-1380 with the desired once-weekly dosing schedule.³¹ The clinical outcomes are consistent with the corresponding preclinical data³³ showing that 5 mg/kg TV-1380 only decreased cocaine (30 μg/kg/infusion) self-administration in monkeys for three days, and it did not completely block the cocaine self-administration in monkeys even on day 1. To effectively keep abstinence from cocaine, TV-1380 would have to be administered more frequently, at least twice per week, for the cocaine dependence treatment. Unfortunately, according to the survey of treatment providers, an injection product for cocaine dependence treatment would be unacceptable if it had to be administered more frequently than once weekly and, hence, the clinical development program with TV-1380 was terminated.³¹ Ideally, it is highly desirable to have a long-acting CocH entity with a biological half-life of at least one week.

To overcome the dosing frequency problem, different CocH delivery approaches have been explored, including development of a viral vector-based gene therapy^{37, 34} or a genome-edited skin epidermal stem cell approach³⁵ to deliver the gene encoding one of previously reported CocHs, using a revised HSA fusion construct,³⁶ and using human immunoglobulin G1 (IgG1) Fc fragment (SEQ ID NO: 2), instead of HSA, as an alternative carrier protein to produce Fc-fused CocH protein, known as CocH3-Fc, with a prolonged biological half-life,³⁷⁻³⁸ but all with limitations. Development of a practically useful viral vector-based gene therapy for cocaine dependence.^{27, 34} will face serious chronic safety concerns due to the use of viral vector. Compared to the viral vector-based gene therapy approach, the genome-edited skin epidermal stem cell approach³⁵ should be much safer, but much less efficient to produce the CocH protein; the average peek plasma CocH concentration (in mice) generated by the genome-edited skin cells was only ˜1 mg/L,³⁵ which is likely too low to completely block the potent psychostimulant effects of cocaine. In addition, the application of the genome-edited skin cell approach to a patient would require two surgeries (for the skin collection and implantation) between genome-editing the skin epidermal skin cells. As noted in commonly assigned U.S. Patent Application Publication No. 2021/0189359, which is incorporated herein by reference, the biological half-life of CocH3 is much shorter than native BChE. Compared to CocH3 itself, previously designed and tested CocH3-Fc was expected to have a longer biological half-life, because the Fc domain can bind with neonatal Fc receptor (FcRn) in the acidic environment of endosome and is later transported to the cell surface where upon exposure to a neutral pH IgG is released back to the main bloodstream.³⁷ But the actual binding affinity of CocH3-Fc with FcRn was not measured. Butyrylcholinesterase fusion protein CocH3-Fc did, however, show a significantly improved biological half-life (t_{1/2}=˜4 days in rats),³⁷ but it is still shorter than the desired one week (7 days).

Thus, there remains a need in the art for butyrylcholinesterase fusion proteins with improved biological half-lives and methods for treating cocaine use disorder using the same.

Furthermore, it is well known that BChE can intercept and destroy organophosphorus (OP) nerve poisons before they reach their target—acetylcholinesterase (AChE).⁵⁶⁻⁵⁸ Thus, administration of BChE is recognized as an effective and safe medication for the prevention of organophosphorus (OP) nerve agent toxicity.⁵⁸⁻⁶¹ Accordingly, the use of butyrylcholinesterase fusion proteins with improved biological half-lives may also prove advantageous in methods of treating organophosphorus toxicity.

### SUMMARY

The presently-disclosed subject matter meets some or all of the above-identified needs, as will become evident to those of ordinary skill in the art after a study of information provided in this document.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently disclosed subject matter includes methods of treating cocaine use disorder in which an effective amount of a butyrylcholinesterase (BChE) fusion protein is administered to a subject in need thereof. The BChE fusion protein comprises a BChE protein and a Fc polypeptide mutant with one or more amino acid substitutions as compared to wild-type Fc (SEQ ID NO: 2). In some embodiments, the Fc polypeptide mutant is selected from the group consisting of: Fc(M6) (SEQ ID NO: 6), Fc(M3) (SEQ ID NO: 7), and Fc(M4) (SEQ ID NO: 8). In one such embodiment, the Fc polypeptide mutant is one of Fc(M6) (SEQ ID NO: 6) and Fc(M4) (SEQ ID NO: 8). In some embodiments, the BChE fusion protein is selected from the group consisting of: CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16), BChE-Fc(M3) (SEQ ID NO: 17), BChE-Fc(M4) (SEQ ID NO: 18), and BChE-Fc(M6) (SEQ ID NO: 19).

In some embodiments, the BChE protein is a BChE mutant with one or more amino acid substitutions as compared to wild-type BChE (SEQ ID NO: 1). In one such embodiment, the BChE mutant is selected from the group consisting of CocH3 (SEQ ID NO: 3), CocH3G (SEQ ID NO: 4), and CocH5G (SEQ ID NO: 5). In some embodiments, the butyrylcholinesterase fusion protein is a sequence selected from the group consisting of CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16).

In some embodiments, administration of the butyrylcholinesterase fusion protein occurs once per week. In some embodiments, administration of the butyrylcholinesterase fusion protein is intravenous or intramuscular. In some embodiments, the subject to which the butyrylcholinesterase fusion protein is administered is a mammal. In some embodiments, the subject to which the butyrylcholinesterase fusion protein is administered is a human. In some embodiments, the butyrylcholinesterase fusion protein is administered in a dose between about 0.075 mg/kg and about 3 mg/kg.

The presently disclosed subject matter also includes methods of treating organophosphorous poisoning in a subject in which an effective amount of a butyrylcholinesterase (BChE) fusion protein is administered to a subject in need thereof. The butyrylcholinesterase fusion protein comprises a BChE protein and a Fc polypeptide mutant with one or more amino acid substitutions as compared to wild-type Fc (SEQ ID NO: 2). In some embodiments, the Fc polypeptide mutant is selected from the group consisting of: Fc(M6) (SEQ ID NO: 6), Fc(M3) (SEQ ID NO: 7), and Fc(M4) (SEQ ID NO: 8). In one such embodiment, the Fc polypeptide mutant is one of Fc(M6) (SEQ ID NO: 6) and Fc(M4) (SEQ ID NO: 8). In some embodiments, the BChE fusion protein is selected from the group consisting of: CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16), BChE-Fc(M3) (SEQ ID NO: 17), BChE-Fc(M4) (SEQ ID NO: 18), and BChE-Fc(M6) (SEQ ID NO: 19).

In some embodiments, the BChE protein is a BChE mutant with one or more amino acid substitutions as compared to wild-type BChE (SEQ ID NO: 1). In one such embodiment, the BChE mutant is selected from the group consisting of CocH3 (SEQ ID NO: 3), CocH3G (SEQ ID NO: 4), and CocH5G (SEQ ID NO: 5). In some embodiments, the butyrylcholinesterase fusion protein is a sequence selected from the group consisting of CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16). In some embodiments, the subject to which the butyrylcholinesterase fusion protein is administered is a mammal.

### BRIEF DESCRIPTION OF THE DRAWINGS

The presently-disclosed subject matter will be better understood, and features, aspects and advantages other than those set forth above will become apparent when consideration is given to the following detailed description thereof. Such detailed description makes reference to the following drawings, wherein:

FIGS. 1A-1C are models showing energy-minimized binding structures of human FcRn with either CocH3-Fc (SEQ ID NO: 9) or CocH3-Fc(M6) (SEQ ID NO: 10). FIG. 1A is a model showing the overall structure of FcRn binding with CocH3-Fc(M6) (SEQ ID NO: 10) dimer. For clarity, highlighted in colored balls are the V530, Y567, T569, E571, E671, and M673 residues from only one subunit of the CocH3-Fc(M6) (SEQ ID NO: 10) dimer. FIG. 1B is a model showing detailed intermolecular interactions of FcRn with CocH3-Fc (SEQ ID NO: 9). FIG. 1C is a model showing detailed intermolecular interactions of FcRn with CocH3-Fc(M6) (SEQ ID NO: 10).

FIG. 2 is a graph showing plots of measured protein binding (%) versus the Fc-fused protein concentration for CocH3-Fc (SEQ ID NO: 9) and CocH3-Fc(M6) (SEQ ID NO: 10), measured in triplicate based on ELISA assay.

FIG. 3 is a graph showing the pharmacokinetic (PK) profile of CocH3-Fc(M6) (SEQ ID NO: 10) after intravenous administration of 0.075 mg/kg CocH3-Fc(M6) (SEQ ID NO: 10) in four rats (n=4).

FIG. 4 is a graph showing the effects of CocH3-Fc(M6) (SEQ ID NO: 10) on cocaine-induced hyperactivity in rats (n=8 per group). 3 mg/kg CocH3-Fc(M6) (SEQ ID NO: 10) or saline was injected intravenously through tail veins 18 days before intraperitoneal injection of 10 mg/kg cocaine or saline.

FIGS. 5A and 5B are graphs showing the enzyme activity of CocH3-Fc (SEQ ID NO: 9) with paraoxon. FIG. 5A shows the enzyme activity of Coch3-Fc (SEQ ID NO: 9) with paraoxon. FIG. 5B shows a comparison of the enzyme activity of CocH3-Fc (SEQ ID NO: 9) and wild-type BChE (SEQ ID NO: 1) with paraoxon under similar conditions, with completed reaction occurring in only 1 hour with CocH3-Fc (SEQ ID No. 9) and after 40 hours with wild-type BChE (SEQ ID NO: 1).

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

The present application can “comprise” (open ended) or “consist essentially of” the components of the present invention as well as other ingredients or elements described herein. As used herein, “comprising” and grammatical variations thereof are open ended and mean the elements recited, or their equivalent in structure or function, plus any other element or elements which are not recited. The terms “having” and “including”, as well as grammatical variations of the same (e.g., “have” or “include”), are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, and in some embodiments ±0.01% from the specified amount, as such variations are appropriate to perform the disclosed methods.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to a target of administration. The subject of the herein disclosed methods can be a mammal. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. A “patient” refers to a subject afflicted with a disease or disorder. The term “patient” includes human and veterinary subjects.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

The term “effective amount” refers to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level if or any particular patient will depend upon a variety of factors including the disorder being treated and the severity of the disorder; the specific composition employed; the age, bodyweight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products.

As used herein, the term “BChE protein” refers to wild-type BChE (SEQ ID NO: 1) or a BChE mutant which has one or more amino acid substitutions as compared to SEQ ID NO: 1. For example, BChE protein includes, but is not necessarily limited to: wild-type BChE (SEQ ID NO: 1); CocH3 (SEQ ID NO: 3); CocH3G (SEQ ID NO: 4); and CocH5G (SEQ ID NO: 5).

In instances where the term “Fc polypeptide mutant” is referred to herein without additional description or characterization as to the nature of mutations present within the polypeptide, such term will be understood to mean a Fc polypeptide mutant having one or more amino acid substitutions as compared to SEQ ID NO: 2.

One embodiment of the present invention is a method of treating cocaine use disorders in a subject, comprising: administering to the subject an effective amount of a butyrylcholinesterase (BChE) fusion protein to a subject in need thereof. The BChE fusion protein comprises a BChE protein and a Fc polypeptide mutant with one or more amino acid substitutions as compared to wild-type Fc (SEQ ID NO: 2). Typically, the C-terminus of the BChE protein will be fused to the N-terminus of the Fc polypeptide mutant.

In some embodiments, the Fc polypeptide mutant is selected from the group consisting of: Fc(M6) (SEQ ID NO: 6), Fc(M3) (SEQ ID NO: 7), and Fc(M4) (SEQ ID NO: 8). In one such embodiment, the Fc polypeptide mutant is one of Fc(M6) (SEQ ID NO: 6) and Fc(M4) (SEQ ID NO: 8). In some embodiments, the BChE fusion protein is selected from the group consisting of: CocH3-Fc (SEQ ID NO: 9), CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16), BChE-Fc(M3) (SEQ ID NO: 17), BChE-Fc(M4) (SEQ ID NO: 18), and BChE-Fc(M6) (SEQ ID NO: 19).

In some embodiments, the BChE protein is a BChE mutant with one or more amino acid substitutions as compared to wild-type BChE (SEQ ID NO: 1). In one such embodiment, the BChE mutant is selected from the group consisting of CocH3 (SEQ ID NO: 3), CocH3G (SEQ ID NO: 4), and CoCH 5G (SEQ ID NO: 5). In some embodiments, the butyrylcholinesterase fusion protein is a sequence selected from the group consisting of CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16).

In some embodiments, administration of the butyrylcholinesterase fusion protein for treatment of a cocaine use disorder occurs once per week. In some embodiments, administration of the butyrylcholinesterase fusion protein occurs once per month. In some embodiments, the butyrylcholinesterase fusion protein is administered intravenously or intramuscularly. In some embodiments, the subject to which the butyrylcholinesterase fusion protein is administered is a mammal. In some embodiments, the subject to which the butyrylcholinesterase fusion protein is administered is a human. In some embodiments, the butyrylcholinesterase fusion protein is administered in a dose between about 0.075 mg/kg and about 3 mg/kg.

Another embodiment of the present invention is a method of treating organophosphorus poisoning in a subject, comprising: administering to the subject an effective amount of a butyrylcholinesterase (BChE) fusion protein to a subject in need thereof. The butyrylcholinesterase fusion protein comprises a BChE protein and a Fc polypeptide mutant with one or more amino acid substitutions as compared to wild-type Fc (SEQ ID NO: 2). In some embodiments, the Fc polypeptide mutant is selected from the group consisting of: Fc(M6) (SEQ ID NO: 6); Fc(M3) (SEQ ID NO: 7), and Fc(M4 (SEQ ID NO: 8). In one such embodiment, the Fc polypeptide mutant is one of Fc(M6) (SEQ ID NO: 6) and Fc(M4) (SEQ ID NO: 8). In some embodiments, the BChE fusion protein is selected from the group consisting of: CocH3-Fc (SEQ ID NO: 9), CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16), BChE-Fc(M3) (SEQ ID NO: 17), BChE-Fc(M4) (SEQ ID NO: 18), and BChE-Fc(M6) (SEQ ID NO: 19). In some embodiments, the BChE protein is a BChE mutant with one or more amino acid substitutions as compared to wild-type BChE (SEQ ID NO: 1). In one such embodiment, the BChE mutant is selected from the group consisting of CocH3 (SEQ ID NO: 3), CocH3G (SEQ ID NO: 4), and CoCH 5G (SEQ ID NO: 5). In some embodiments, the butyrylcholinesterase fusion protein is a sequence selected from the group consisting of CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16). In some embodiments, the subject to which the butyrylcholinesterase fusion protein is administered is a mammal.

In some embodiments, administration of the BChE fusion protein for treatment of organophosphorous poisoning occurs once per week. In some embodiments, administration of the BChE fusion protein occurs once per month. In some embodiments, the BChE fusion protein is administered intravenously or intramuscularly. In some embodiments, the subject to which the BChE fusion protein is administered is a mammal. In some embodiments, the subject to which the BChE fusion protein is administered is a human. In some embodiments, the BChE is administered in a dose between about 0.075 mg/kg and 3 mg/kg.

### EXAMPLES

Materials and Methods

Molecular modeling. The structure of the BChE fusion protein CocH3-Fc (SEQ ID NO: 9) was modeled using PyMol software³⁹ starting from the X-ray crystal structures of human BChE (PDB 4BDS)⁴⁰ and FcRn-Fc complex (PDB 4N0U).⁴¹ The modeled CocH3-Fc structure was superimposed to the X-ray crystal structure of human FcRn-Fc complex (PDB 4NOU)⁴¹ in order to obtain the initial structure of the complex of human FcRn and CocH3-Fc (SEQ ID NO: 9). All the ionizable residues were set to the standard protonated or deprotonated states at pH 6. Notably, all the histidine residues were protonated under pH 6. The whole complex structure was then solvated in an orthorhombic box consisting of 88,495 TIP3P water⁴² molecules, with a minimal distance of 10 Å from the protein to the box boundary. The whole system was neutralized by adding 21 chloride counter ions with a size of 164 Å×132 Å×116 Å. After the whole system was set up, a series of energy-minimization calculations were carried out by using the Sander module of the Amber 16 program⁴³ with a non-bonded cut-off of 10 Å and the conjugate gradient energy-minimization method.

Starting from energy-minimized complex structure of CocH3-Fc binding with FcRn, various possible mutations on CocH3-Fc (SEQ ID NO: 9) were checked visually, leading to identification of favorable CocH3-Fc mutants (corresponding to SEQ ID NO: 10, SEQ ID NO: 11, SEQ ID NO: 12, SEQ ID NO: 13, SEQ ID NO: 14, SEQ ID NO: 15, and SEQ ID NO: 16) and other BChE fusion proteins (corresponding to SEQ ID NO: 17, SEQ ID NO: 18, and SEQ ID NO: 19) as shown in Table 3 below for further computational modeling using the same procedure described above. As shown in Table 1 and Table 3 below, each of the BChE fusion proteins examined included (i) wild-type BChE (SEQ ID NO: 1) or a BChE mutant thereof corresponding to CocH3 (SEQ ID NO: 3), CocH3G (SEQ ID NO: 4), or CocH5G (SEQ ID NO: 5) and (ii) a mutant of wild-type Fc (SEQ ID NO: 2) (i.e., a Fc polypeptide mutant) corresponding to Fc(M6) (SEQ ID NO: 6), Fc(M3) (SEQ ID NO: 7), or Fc(M4) (SEQ ID NO: 8). Finally, the molecular mechanics-generalized Born and surface area continuum solvation (MM-GBSA) method⁴⁴ was used to estimate the binding free energies of FcRn binding with CocH3-Fc(M6) (SEQ ID NO: 10) and CocH3-Fc (SEQ ID NO: 9) (control for the calculated binding free energy), as shown in Table 2 below.

TABLE 1
List of SEQ ID Proteins and Mutations

| Protein | Mutations | Sequence ID |
|---|---|---|
| BChE wt | None | SEQ ID NO: 1 |
| Fc wt | None | SEQ ID NO: 2 |
| CocH3 | A199S/F227A/S287G/A328W/Y332G | SEQ ID NO: 3 |
| CocH3G | A199S/F227A/S287G/A328W/Y332G/T523G | SEQ ID NO: 4 |
| CocH5G | A199S/F227A/P285A/S287G/A328W/Y332G/T523G | SEQ ID NO: 5 |
| Fc(M6) | A530V/M567Y/S569T/T571E/D671E/L673M | SEQ ID NO: 6 |
| Fc(M3) | A530V/D671E/L673M | SEQ ID NO: 7 |
| Fc(M4) | A530V/M567Y/D671E/L673M | SEQ ID NO: 8 |

Materials for experimental studies. The cDNA for CocH3-Fc (SEQ ID NO: 9) was generated in a previous study.³⁷ Plasmid pFUSE-hIgG1-Fc2 was purchased from InvivoGen (San Diego, CA). Protein expression vector pCMV-MCS was ordered from Agilent (Santa Clara, CA), and pCSC-SP-PW lentiviral vector (plasmid 12335), pMDLg/pRRE (plasmid 12251), pRSV-Rev (plasmid 12253), and pCMV-VSV-G (plasmids 8454) were obtained from Addgene (Cambridge, MA). Phusion DNA polymerase, restriction endonucleases, and T4 DNA ligase were ordered from New England Biolabs (Ipswich, MA). DpnI endonuclease was obtained from Thermo Fisher Scientific (Waltham, MA). Gen synthesis for human FcRn was ordered from GenScript (Piscataway, NJ), and all other oligonucleotides were synthesized by Eurofins MWG Operon (Huntsville, AL). Chinese Hamster Ovary-suspension (CHO-S) cells, Human Embryonic Kidney (HEK)-293FT, FreeStyle™ CHO Expression Medium, HT Supplement, L-glutamine, Dulbecco's Modified Eagle's Medium (DMEM), Fetal Bovine Serum (FBS), 4-12% Tris-Glycine Mini Protein Gel, and SimpleBlue SafeStain were from Invitrogen (Grand Island, NY). TransIT-PRO® Transfection Kit was obtained from Mirus (Madison, WI). The rmp Protein A Sepharose Fast Flow was purchased from GE Healthcare Life Sciences (Pittsburgh, PA). Centrifugal Filter Units were obtained from Millpore (Billerica, MA). (−)-Cocaine was provided by the National Institute on Drug Abuse (NIDA) Drug Supply Program (Bethesda, MD), and [³H](−)-Cocaine (50 Ci/mmol) was ordered from PerkinElmer (Waltham, Massachusetts). All other general chemicals were purchased from Thermo Fisher Scientific (Waltham, MA) or Sigma-Aldrich (St. Louis, MO).

Male Sprague-Darley rats (200-275 g) were ordered from Harlan (Harlan, Indianapolis, IN). All the animal experiments were conducted in the animal laboratories of the University of Kentucky's Division of Laboratory Animal Resources (DLAR) facility (PHS assurance number A3336-01; USDA number 61-R-0002; AAALAC, Intl. Unit #13). The animal procedures used in this project were approved by the Institutional Animal Care and Use Committee (IACUC). Veterinary care and animal husbandry were provided and supervised by the staff of the DLAR facility. All animals were housed in clean, adequately-sized, stainless-steel cages at 21-22° C. and were allowed ad libitum access to food and water for 1 week before any experiments. They were monitored daily by the study staff and by members of the veterinary staff for general health and to detect signs of discomfort due to testing and/or the administration of drugs.

Protein preparation. In order to prepare the newly designed protein CocH3-Fc(M6) (SEQ ID NO: 10), the cDNA for the CocH3-Fc(M6) was first constructed starting from the cDNA for the CocH3-Fc in a pCMV-MCS expression plasmid.³⁷ The site-directed mutagenesis was performed by using the QuikChange method,⁴⁵ wherein a pair of complementary mutagenic primers were used to amplify the entire plasmid in a thermocycling reaction using pfu polymerase. Further, in order to express the CocH3-Fc(M6) (SEQ ID NO: 10) protein in large-scale, a previously described lentivirus-based method (using the pCSC-SP-PW lentiviral vector)^{37, 46} was performed to generate a high-efficient stable (CHO-S) cell line expressing CocH3-Fc(M6). The obtained stable CHO-S cell pool was kept frozen before being used for large-scale protein production.

The large-scale production of CocH3-Fc(M6) (SEQ ID NO: 10) was performed in an agitated bioreactor BioFlo/CelliGen 115 (Eppendorf, Hauppauge, NY). Before being transferred into the bioreactor, cells grew at 37° C. in shake flasks until to designated volume and density. On the day of transferring, cells in shake flasks were centrifuged at 1500 rpm for 5 minutes at room temperature, resuspended in fresh culture medium, and transferred into the bioreactor. CO₂/air gas overlay was set such that the pH of cell culture medium was maintained at 7.0-8.0. The bioreactor was run in a batch model with a temperature of 32° C. After 10 days, the culture medium was harvested, and the protein was purified by using the protein A affinity chromatography³⁷ with an ÄKTA Avant 150 system (GE Healthcare Life Sciences, Pittsburgh, PA). The purified protein was dialyzed in storage buffer and stored at −80° C. before use.

Human FcRn was prepared as a soluble single-chain fusion protein as described by Feng et al.⁴⁷ In the protein, the beta 2 microglobulin chain (B2M) was genetically linked to the N-terminus of the transmembrane domain-truncated heavy chain through a short amino-acid linker.⁴⁷ It has been demonstrated⁴⁷ that the generated soluble single-chain fusion FcRn is fully functional, can be highly expressed in mammalian cells, and can be purified easily through simple affinity chromatography via its C-terminal hexa-histidine tag. In light of the encouraging outcomes,⁴⁷ the C-terminus of the B2M chain was linked to the N-terminus of the heavy chain (without the transmembrane domain) using (GGGGS)₃ as the linker. A hexa-histidine tag (6×His) was introduced at the C-terminus of the fusion protein. The fusion gene for the single-chain FcRn was cloned into a mammalian expression vector, pCMV-MCS. The FcRn protein was expressed in HEK-293 cells and then purified by immobilized metal ion affinity chromatography.¹⁸ The purified protein was dialyzed in a storage buffer and stored at −80° C. before use.

cDNA construction, expression, and large-scale production of the other CocH3-Fc mutants—CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3 (SEQ ID NO: 14), CocH5G-Fc(M4) (SEQ ID NO: 15), and CocH5G-Fc(M6) (SEQ ID NO: 16)—and BChE fusion proteins corresponding to BChE-Fc(M3) (SEQ ID NO: 17), BChE-Fc(M4) (SEQ ID NO: 18), and BChE-Fc(M6) (SEQ ID NO: 19) was performed in the same manner as described above with reference to CocH3-Fc(M6) (SEQ ID NO: 10).

In vitro binding affinity assay. The binding affinity of the BChE fusion proteins corresponding to CocH3-Fc (SEQ ID NO: 9), CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), and CocH5G-Fc(M6) (SEQ ID NO: 16), BChE-Fc(M3) (SEQ ID NO: 17), BChE-Fc(M4) (SEQ ID NO: 18), and BChE-Fc(M6) (SEQ ID NO: 19) with FcRn was determined by using an enzyme-linked immunosorbent assay (ELISA). 400 ng of the 6×His-tagged FcRn in 100 μL 0.05 M PBS, pH 7.4, was immobilized in a 96-well flat-bottomed EIA plate (Corning) at 4° C. overnight (or 37° C. for 2 hours). At the same time, corresponding empty wells without FcRn coating were left as a negative control. The liquid was dumped from the plates and the rest was drained on a paper towel. Coated wells were blocked with blocking buffer (0.05 M PBS, pH 6.0, containing 1 mg/ml casein) (250 μL/well) at room temperature (RT) for 1 hour. After washing twice with washing buffer (0.05 M PBS, pH 6.0) (250 μL/well), 100 μL of BChE fusion protein diluted in blocking buffer, pH 6.0 was added to each well at a range of concentrations. The plate was then covered with an adhesive plastic and incubated, with continuous shaking, at RT for 1 hour. After washing three times with washing buffer, an enzyme horseradish peroxidase (HRP)-conjugated antibody (anti-human IgG-Fc Ab-HRP) (70 μL/well), diluted with blocking buffer at a ratio of 1:20,000, was added into each well and incubated at RT for 30 minutes on a shaker. The wells were then washed three times with washing buffer (250 μL/well) before 250 μL TMB substrate was added to the wells. The ELISA plate was kept in the dark until the desired color developed. The reaction was stopped with 100 μL of 0.5 M HCl. The absorbance (the developed blue color) was measured at 450 nm using a microplate reader. All measurements were performed in triplicate or quadruplicate.

Ex vivo cocaine hydrolysis and PK analysis. Wild-type rats (n=4) were administered intravenously (IV) with a dose of 0.075 mg/kg CocH3-Fc(M6) (SEQ ID NO: 10), followed by daily blood sampling at 1 hour, 4 hours, 8 hours, 12 hours, 1 day, and once every day until Day 30 after the enzyme administration (FIG. 3). Blood samples were taken from saphenous vein puncture using a needle. Each time, approximately 50-80 μL blood was collected into a heparin-coated capillary tube. Collected blood samples were centrifuged for 15 min. at a speed of 5000 g to separate the plasma, which was kept at 4° C. before analysis. A sensitive radiometric assay^{20, 32} with 100 μM (−)-cocaine was used to measure the enzyme activity (ex vivo pharmacodynamics) in plasma and convert the ex vivo cocaine hydrolysis activity to the concentration of the enzyme: [E]=Vₘₐₓ/k_{cat}. The time-dependent active enzyme concentrations ([E]ₜ) were fitted to a well-known double-exponential equation⁴⁸ by using the GraphPad Prism 7: ([E]ₜ=Ae^{-k}^{ₜ}^{t}+Be^{-k}^{ₜ}^{t}) which accounts for both the enzyme distribution process (the fast phase, associated with k₁) and elimination process (the slow phase, associated with k₂). The half-life (t_{1/2}) associated with the enzyme elimination rate constant k₂ is known as the elimination half-life or biological half-life. The half-life of the of the other CocH3-Fc mutants—CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3 (SEQ ID NO: 14), CocH5G-Fc(M4) (SEQ ID NO: 15), and CocH5G-Fc(M6) (SEQ ID NO: 16)—and BChE fusion proteins corresponding to BChE-Fc(M3) (SEQ ID NO: 17), BChE-Fc(M4) (SEQ ID NO: 18), and BChE-Fc(M6) (SEQ ID NO: 19) was determined in the same manner as described for CocH3-Fc(M6) (SEQ ID NO: 10).

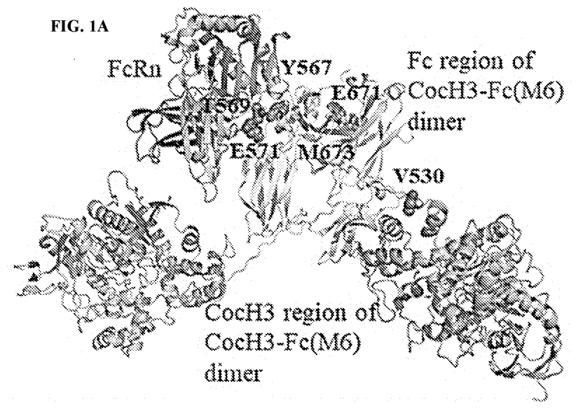

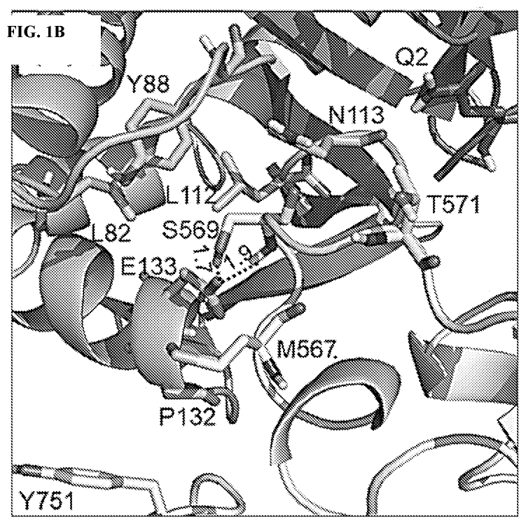

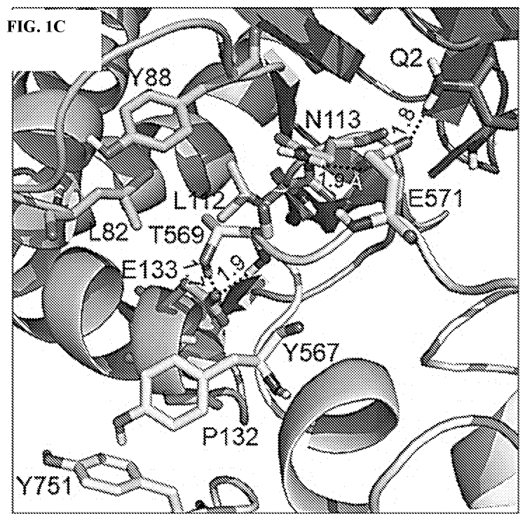

It should be noted that human FcRn (hFcRn) transgenic animal models⁴⁹ are popularly used in PK studies on Fc fused proteins. The use of wild-type rats was elected for a number of reasons. First, the key residues (including Q2, L112, N113, P132, and E133 indicated in FIG. 1B and FIG. 1C) of hFcRn that directly interact with the mutated residues (including Y567, T569, and E571 indicated in FIG. 1C) of the Fc are exactly the same as those of rat FcRn (rFcRn). For this reason, the same mutations are expected to similarly improve the binding of the BChE fusion protein with both hFcRn and rFcRn. In addition, previous PK studies on CocH3-Fc were also carried out in the same strain of wild-type rats.³⁷ As such, the PK data may be conveniently compared without unnecessarily repeating the PK study on CocH3-Fc.

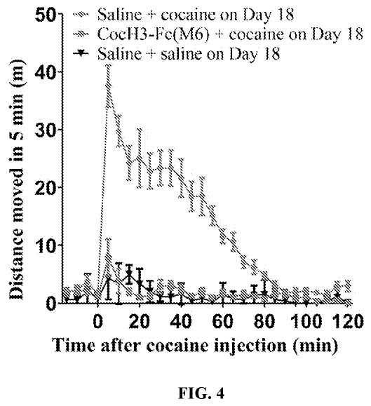

Locomotor activity and toxicity testing. The effects of CocH3-Fc(M6) (SEQ ID NO: 10) on cocaine-induced hyperactivity was evaluated by performing locomotor activity testing in three groups of rats (n=8 per group), along with daily inspection of any possible toxicity or discomfort/uneasiness signs (or any possible abnormal behaviors) within four weeks after the enzyme administration (FIG. 4). Rats were injected intravenously (IV) with a single dose of CocH3-Fc(M6) (SEQ ID NO: 10) (3 mg/kg, for the treatment group) or saline (for the two control groups), followed by locomotor activity testing on Day 18 with intraperitoneal (IP) injection of 20 mg/kg cocaine (for the treatment group and positive control group) or saline (for the negative control group). It has been known that the commonly used oral doses of cocaine in humans are 50-300 mg (or ˜1-5 mg/kg for an average body weight of 60 kg);⁵⁰ the minimum lethal dose of cocaine was estimated to be 1.2 g or ˜20 mg/kg for an average body weight of 60 kg (http://drug.addictionblog.org/cocaine-overdose-how-much-amount-of-cocaine-to-overdose/). So, the dose of 20 mg/kg cocaine reflects the high end of typically abused doses of cocaine in humans.

The locomotor activity was monitored in high density, non-porous plastic chambers measuring 50 cm (L)×50 cm (W)×38 cm (H) in a light- and sound-attenuating behavioral test enclosure (San Diego Instruments, San Diego, CA). Cumulative horizontal distance traveled was recorded by EthoVision XT video tracking system (Noldus Information Technology, Wageningen, Netherlands) to represent the locomotor activity. The hyperactivity was reflected by the increased horizontal distance traveled.

Results

Molecular interaction with FcRn. The CocH3-Fc mutant design started from molecular modeling of CocH3-Fc (SEQ ID NO: 9) binding with FcRn at pH 6 based on the observation that an IgG (or a Fc fused protein³⁷) has a long biological half-life because the Fc region of the protein can bind with FcRn in an acidic environment (pH ˜6) of endosome and later the protein is transported to the cell surface to be released back to the main bloodstream under a neutral pH.⁵¹ It is thus reasonable to assume that a CocH3-Fc mutant with an improved binding affinity to FcRn at pH 6 would have a prolonged biological half-life. CocH3-Fc (SEQ ID NO: 9) is a fusion protein consisting of 762 amino-acid residues, including the first 529 residues of CocH3 (A199S/F227A/S287G/A328W/Y332G mutant of human BChE) on the N-terminus plus the first 233 residues (Fc region) of human IgG1. According to the molecular modeling, the A530V/M567Y/S569T/T571E/D671E/L673M mutant (see FIG. 1A) of CocH3-Fc appears to have a markedly improved binding affinity with FcRn.

Specifically, the energy-minimized structures of FcRn binding with CocH3-Fc (SEQ ID NO: 9) and CocH3-Fc(M6) (SEQ ID NO: 10) are depicted in FIGS. 1B and 1C, respectively. As seen in the energy-minimized protein-protein binding structures depicted in FIGS. 1B and 1C, changing amino-acid residue T571 to E will produce a favorable hydrogen bond between an oxygen atom of the carboxylate group on the E571 side chain and the amine group on the side chain of residue Q2 of FcRn. Changing S569 to T adds an additional methyl group to enhance the hydrophobic interaction with the hydrophobic residues (L82, Y88, and L112) of FcRn. Similarly, changing M567 to Y will also add a phenyl ring on the Y567 side chain to slightly enhance the hydrophobic interaction with the hydrophobic environment of FcRn. The remaining three residues mutated (including V530, E671, and M673) have no direct contacts with any residues of FcRn, as shown in FIG. 1A, but they indirectly improve the binding affinity with FcRn based on the binding free energy calculations. According to the MM-GBSA binding free energy calculations, the directly calculated binding free energies (without any empirical corrections) at 25° C. (298 K) for FcRn binding with CocH3-Fc (SEQ ID NO: 9) and CocH3-Fc(M6) (SEQ ID NO: 10) were −62.9 and −94.2 kcal/mol (Table 2), respectively, predicting that CocH3-Fc(M6) (SEQ ID NO: 10) should have a markedly higher binding affinity with FcRn compared to CocH3-Fc (SEQ ID NO: 9). As is well known, the binding free energies obtained from the MM-GBSA calculations are reasonable only for the relative magnitudes of different binding systems. But the directly obtained MM-GBSA binding free energy data should be corrected empirically for quantitative predictions.⁵² Specifically, assuming K_{d}=˜4 μM (see below for the experimental estimate) for CocH3-Fc (SEQ ID NO: 9) binding with FcRn, the binding free energy should be −7.3 kcal/mol according to the well-known thermodynamic equation. Hence, the correction factor (denoted as w) should be −62.9/(−7.3)=8.56 or w=8.56. Applying this correction factor to CocH3-Fc(M6) (SEQ ID NO: 10) binding with FcRn, −94.2/w=−11.0 kcal/mol was obtained (Table 2).

TABLE 2
Computationally Estimated and Experimental Binding Free
Energies of CocH3-Fc and CocH3-Fc(M6) with FcRn

| | ΔG_{d} (kcal/mol, MM-GBSA) | | Experimental data | | |
|---|---|---|---|---|---|
| | Uncorrected | Corrected | | | ΔG_{d} |
| Fc-fused protein | ΔG_{d} ^{a} | ΔG_{d} ^{b} | K_{d} | | (kcal/mol) ^{c} |
| CocH3-Fc | −62.9 | −7.3 | ~4 | μM | −7.3 |
| (SEQ ID NO: 9) | | | | | |
| CocH3-Fc(M6) | −94.2 | −11.0 | 43 | nM | −10.0 |

(SEQ ID NO: 10)

^{a} The MM-GBSA-calculated binding free energy without any empirical correction.
^{b} Corrected binding free energy was obtained by using an empirical correction factor (w = 8.56): ΔG_{d}(corrected) = ΔG_{d}(uncorrected)/w to fit the experimental ΔG_{d} value of −7.3 kcal/mol (corresponding to K_{d} =~4 μM).
^{c} Experimental binding free energy was converted from the measured K_{d} by using thermodynamic equation ΔG_{d} = −R7In K_{d}.

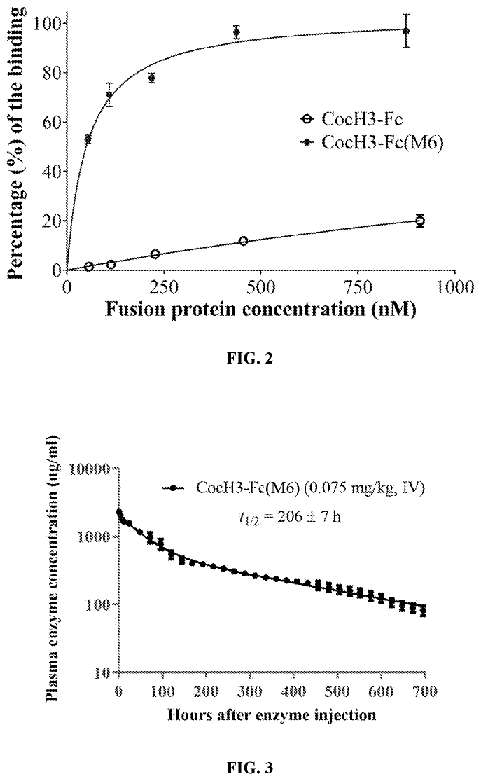

Binding affinity with FcRn. Based on the in vitro binding assays, the fusion proteins including the A530V/M567Y/S569T/T571E/D671E/L673M Fc polypeptide mutation (i.e., the BChE fusion proteins including Fc(M6) (SEQ ID NO: 6)), indeed exhibited a markedly higher binding affinity with FcRn at pH 6. For example, as shown in FIG. 2, CocH3-Fc(M6) (SEQ ID NO: 10) had a high binding affinity, with a dissociation constant (K_{d}) of 43 nM, at pH 6, without changing the catalytic activity against cocaine (in vitro enzyme activity data not shown). In comparison, K_{d}>>1 μM (1000 nM) for CocH3-Fc (SEQ ID NO: 9) under the same experimental conditions; the best estimate was K_{d}=˜4 μM for CocH3-Fc binding with FcRn based on the data indicated in Table 2. So, with respect to CocH3-Fc(M6) (SEQ ID NO: 10) the mutations contained therein markedly improved the binding affinity (K_{d}) from ˜4 μM to 43 nM (an ˜100-fold improvement in the binding affinity). Notably, the computationally predicted binding free energy of −11.0 kcal/mol for CocH3-Fc(M6) (SEQ ID NO: 10) binding with FcRn is close to the experimentally derived binding free energy of −10.0 kcal/mol (corresponding to K_{d}=43 nM), as summarized in Table 2. The other BChE fusion proteins including the Fc polypeptide mutant Fc(M6) (SEQ ID NO: 6)—i.e., CocH3G-Fc(M6) (SEQ ID NO: 13), CocH5G-Fc(M6) (SEQ ID NO: 16), and BChE-Fc(M6) (SEQ ID NO: 19)—also exhibited improved binding affinity as compared to CocH3-Fc (SEQ ID NO: 9), with a dissociation constant (K_{d}) of 40 nM. As shown in Table 3 below, the BChE fusion proteins including Fc polypeptide mutations Fc(M3) and Fc(M4)—i.e., CocH3G-Fc(M3) (SEQ ID NO: 11), CocH3G-Fc(M4) (SEQ ID NO: 12), CocH5G-Fc(M3) (SEQ ID NO: 14), CocH3G-Fc(M4) (SEQ ID NO: 15), BChE-Fc(M3) (SEQ ID NO: 17), and BChE-Fc(M4) (SEQ ID NO: 18)—also exhibited improved binding affinity as compared to CocH3-Fc (SEQ ID NO: 9) with dissociation constants (K_{d}) of 99 nM and 320 nM, respectively.

TABLE 3
Binding Affinities of CocH3-Fc Mutants with FcRn and Their Biological Half-lives in Rats

| BChE Fusion | | | | t_{1/2} |
|---|---|---|---|---|
| Protein | Mutations | K_{d} | | (hr) |
| CocH3-Fc | A199S/F227A/S287G/A328W/Y332G (SEQ ID NO: 9) | ~4 | μM | 87 |
| CocH3-Fc(M6) | A199S/F227A/S287G/A328W/Y332G/A530V/M567Y/ | 43 | nM | 206 |
| | S569T/T571E/D671E/L673M (SEQ ID NO: 10) | | | |
| CocH3G- | A199S/F227A/S287G/A328W/Y332G/T523G/A530V/ | 990 | nM | 110 |
| Fc(M3) | D671E/L673M (SEQ ID NO: 11) | | | |
| CocH3G- | A199S/F227A/S287G/A328W/Y332G/T523G/A530V/ | 320 | nM | 200 |
| Fc(M4) | M567Y/D671E/L673M (SEQ ID NO: 12) | | | |
| CocH3G- | A199S/F227A/S287G/A328W/Y332G/T523G/A530V/ | 40 | nM | 220 |
| Fc(M6) | M567Y/S569T/T571E/D671E/L673M (SEQ ID NO: 13) | | | |
| CocH5G- | A199S/F227A/P285A/S287G/A328W/Y332G/T523G/ | 990 | nM | 110 |
| Fc(M3) | A530V/D671E/L673M (SEQ ID NO: 14) | | | |
| CocH5G- | A199S/F227A/P285A/S287G/A328W/Y332G/T523G/ | 320 | nM | 200 |
| Fc(M4) | A530V/M567Y/D671E/L673M (SEQ ID NO: 15) | | | |
| CocH5G- | A199S/F227A/P285A/S287G/A328W/Y332G/T523G/ | 40 | nM | 220 |
| Fc(M6) | A530V/M567Y/S569T/T571E/D671E/L673M | | | |
| | (SEQ ID NO: 16) | | | |
| BChE-Fc(M3) | A530V/D671E/L673M (SEQ ID NO: 17) | 990 | nM | 110 |
| BChE-Fc(M4) | A530V/M567Y/D671E/L673M (SEQ ID NO: 18) | 320 | nM | 200 |
| BChE-Fc(M6) | A530V/M567Y/S569T/T571E/D671E/L673M | 40 | nM | 220 |

(SEQ ID NO: 19)

Ex vivo cocaine hydrolysis activity and PK profile. The BChE fusion proteins of CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16), BChE-Fc(M3) (SEQ ID NO: 17), BChE-Fc(M4) (SEQ ID NO: 18), and BChE-Fc(m6) (SEQ ID NO: 19) were evaluated for their actual ex vivo cocaine hydrolysis activity and the activity-based PK profile in rats. In this regard, rats were administered intravenously (IV) with a dose (0.075 mg/kg) of a BChE fusion protein, followed by blood sampling at various time points after the enzyme administration, as shown, e.g., by the testing of CocH3-Fc(M6) (SEQ ID NO: 10) in FIG. 3. During the in vivo studies on the CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16), BChE-Fc(M3) (SEQ ID NO: 17), BChE-Fc(M4) (SEQ ID NO: 18), and BChE-Fc(M6) (SEQ ID NO: 19) in rats, it was desired to ascertain whether IV administration of the enzyme can generate active enzyme capable of hydrolyzing cocaine in plasma. So, using an ex vivo cocaine hydrolysis assay,³⁶ it was validated that the enzyme in the collected rat plasma after the administration the BChE fusion protein was indeed able to accelerate cocaine hydrolysis ex vivo. Then, the measured ex vivo cocaine hydrolysis activity (Vₘₐₓ) was converted to the active enzyme concentration ([E]) by using the known catalytic rate constant (k_{cat}=5700 min⁻¹ at 25° C.): [E]=Vₘₐₓ/k_{cat}. Depicted in FIG. 3 is the plot of the determined [E] vs the time for CocH3-Fc(M6) (SEQ ID NO: 10). As shown in FIG. 3, CocH3-Fc(M6) (SEQ ID NO: 10) had a markedly prolonged biological half-life (t_{1/2}=206±7 hours or ˜9 days).⁵³ In addition to the ex vivo cocaine hydrolysis activity-based PK assay, an attempt was made to determine the total CocH3-Fc(M6) (SEQ ID NO: 10) protein concentrations in the plasma samples by performing a human IgG (hIgG) Fc-specific immunoassay using an anti-hIgG (Fc-specific) antibody⁵³ in the initial stage of our PK study, but found no significant difference between the two types of PK data. Hence, it was finally elected to solely use the more sensitive ex vivo cocaine hydrolysis activity-based assay for the PK study.

The other BChE fusion proteins tested including the Fc polypeptide Fc(M6) mutant-CocH3G-Fc(M6) (SEQ ID NO: 13), CocH5G-Fc(M6) (SEQ ID NO: 16), and BChE-Fc(M6) (SEQ ID NO: 19)—also had a markedly prolonged biological half-life of about 220 hours (˜9 days) (Table 3). The BChE fusion proteins including Fc polypeptide mutants Fc(M3) and Fc(M4)—CocH3G-Fc(M3) (SEQ ID NO: 11), CocH3G-Fc(M4) (SEQ ID NO: 12), CocH5G-Fc(M3) (SEQ ID NO: 14), CocH3G-Fc(M4) (SEQ ID NO: 15), BChE-Fc(M3) (SEQ ID NO: 17), and BcHE-Fc(M4) (SEQ ID NO: 18)—also exhibited prolonged biological half-life (t_{1/2}) as compared to CocH3-Fc with half-lives of about 110 hours (˜5 days) and 200 hours (˜8 days), respectively (Table 3).

Safety and effect of CocH3-Fc(M6) on cocaine-induced hyperactivity. According to previous in vivo studies on other CocH entities with markedly shorter biological half-lives,³⁶⁻³⁷ a given CocH (with a biological half-life t_{1/2}) at an IV dose of 3 mg/kg could effectively block 20 mg/kg cocaine-induced hyperactivity for a period of 2t_{1/2}. It was thus examined whether IV administration of 3 mg/kg CocH3-Fc(M6) (SEQ ID NO: 10) (with t_{1/2}=˜9 days based on the above-discussed PK study at a dose of 0.075 mg/kg) were able to effectively block 20 mg/kg cocaine-induced hyperactivity on Day 18 after the enzyme administration. As shown in FIG. 4, for the group of rats treated with CocH3-Fc(M6) (SEQ ID NO: 10), on Day 18 after the enzyme administration, IP administration of 20 mg/kg cocaine did not induce significant hyperactivity. It can therefore be concluded that a single dose (3 mg/kg) of CocH3-Fc(M6) (SEQ ID NO: 10) is capable of effectively blocking the cocaine-induced hyperactivity for at least 18 days. Prior to the current study, a dose of the previously reported best available cocaine hydrolase (with t_{1/2}=107 h)³⁷ was able to completely block cocaine-induced hyperactivity for only 9 days.

Besides the locomotor activity testing, the acute toxicity of the enzyme was examined by detecting any possible toxic signs everyday within four weeks after the IV administration of CocH3-Fc(M6) (SEQ ID NO: 10). No toxic signs or adverse effects in any rats were noticed while demonstrating the desired high catalytic efficiency against cocaine, which further suggests that the enzyme CocH3-Fc(M6) (SEQ ID NO: 10) may serve as a promising therapeutic candidate for cocaine dependence treatment.

Discussion

The present disclosure relates to rationally designed long-acting BChE fusion proteins including a Fc polypeptide mutant based on combined computational modeling and experimental measurement of the BChE fusion protein with FcRn. Using this strategy, BChE fusion proteins with markedly prolonged biological half-lives as compared to CocH3-Fc (SEQ ID NO: 9) were produced. It should be noted that, for a Fc-fused protein drug such as abatacept,⁵⁴ its biological half-life in humans (e.g. t_{1/2}=12-23 days for abatacept) is usually longer than that (e.g. t_{1/2}=3-6 days for abatacept) in rats by about 3 to 4-fold. For this reason, the change in the biological half-life of BChE fusion proteins corresponding to CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16), BChE-Fc(M3) (SEQ ID NO: 17), BChE-Fc(M4) (SEQ ID NO: 18), and BChE-Fc(M6) (SEQ ID NO: 19) disclosed herein from rats to humans could be similar to that of abatacept. If this is the case, the biological half-life in humans should be much longer than the ˜8 days for the BChE fusion proteins including the Fc polypeptide mutant Fc(M4) (CocH3G-Fc(M4) (SEQ ID NO: 12), CocH5G-Fc(M4) (SEQ ID NO: 15), and BChE-Fc(M4) (SEQ ID NO: 18)) and the ˜9 days for the BChE fusion proteins including the Fc polypeptide mutant Fc(M6) (CocH3-Fc(M6) (SEQ ID NO: 10), CocH3G-Fc(M6) (SEQ ID NO: 13), CocH5G-Fc(M6) (SEQ ID NO: 16), and BChE-Fc(M6) (SEQ ID NO: 19)) observed in rats. Thus, the duration of 3 mg/kg the BChE fusion proteins corresponding to CocH3-Fc(M6) (SEQ ID NO: 10), CocH3G-Fc(M3) (SEQ ID NO: 11), CocH3G-Fc(M4) (SEQ ID NO: 12), CocH3G-Fc(M6) (SEQ ID NO: 13), CocH5G-Fc(M3) (SEQ ID NO: 14), CocH5G-Fc(M4) (SEQ ID NO: 15), CocH5G-Fc(M6) (SEQ ID NO: 16), BChE-Fc(M3) (SEQ ID NO: 17), BChE-Fc(M4) (SEQ ID NO: 18), and BChE-Fc(M6) (SEQ ID NO: 19) action in humans could be much longer than that in rats.

Notably, comparing the BChE fusion proteins disclosed herein which included Fc polypeptide mutant Fc(M6), Fc polypeptide mutant Fc(M3), or Fc polypeptide mutant Fc(M4) with other FDA-approved Fc-fusion protein therapeutics, such as abatacept and alefacept,⁵⁵ the BChE fusion proteins including Fc(M6), Fc(M3), or Fc(M4) have a markedly longer biological half-life in the same species (rats). The same protein redesign strategy described herein could also be valuable in development of other therapeutic proteins with a markedly prolonged biological half-life.

Organophosphorous Poisoning

Organophosphorous (OP) poisoning is due to OP's irreversible inhibition of endogenous AChE and BChE in the body. AChE/BChE will react with OP compound; both the enzyme and OP will be destroyed as a result of the covalent binding. The ratio of OP to AChE or BChE is 1:1, that is each enzyme molecule will destroy one OP molecule. Recombinant wild-type BChE is known to be effective for treatment of organophosphorus poisoning and is expected to have the same reaction rate with OP compared to endogenous BChE. Wild-type BChE only works, however, for a very short period of time due to its short biological half-life (˜8 hours for the recombinant wild-type BChE). As evidenced above, the rationally designed mutations of the Fc portion of the BChE fusion proteins disclosed herein both improved binding affinity with FcRn at pH 6 and substantially prolonged biological half-life as compared to CocH3-Fc (SEQ ID NO: 9).

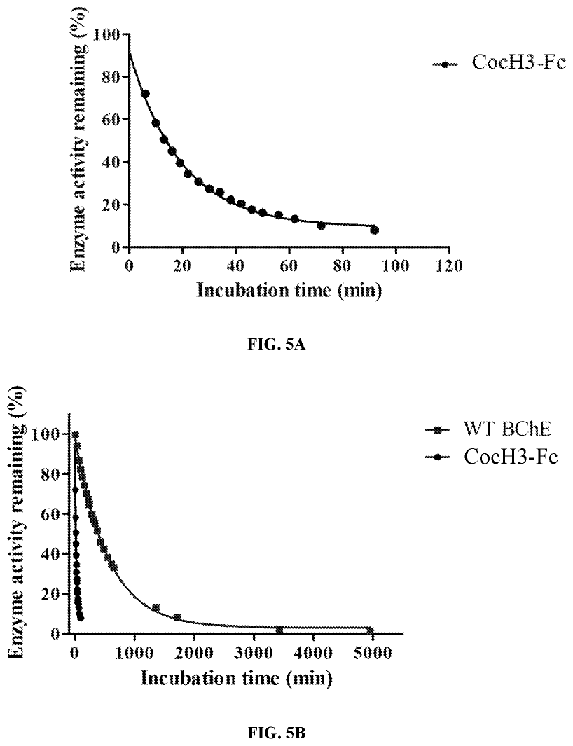

To test the reactivity of CocH3-Fc (SEQ ID NO: 9) with OPs to compete with endogenous BChE and AChE and react with OPs so as to protect a subject from OP poisoning CocH3-Fc and wild-type BChE were examined for their reactivity with paraoxon (a representative OP) in vitro at the same time under the same experimental conditions including the same enzyme concentration (˜5 nM) and same room temperature. Reacting with paraoxon, the enzyme would be inactivated. So, the faster the reaction of the enzyme with paraoxon, the more quickly the enzyme activity decreased. FIG. 5A demonstrates that BChE fusion protein CocH3-Fc (SEQ ID NO: 9) successfully reacted with paraoxon (a representative OP). Further, as shown in FIG. 5B, CocH3-Fc (SEQ ID NO: 9) reacted much more quickly with paraoxon, with completed reaction occurring in only 1 hour, than wild-type BChE (SEQ ID NO: 1), which under similar conditions had a completed reaction with paraoxon after 40 hours. In this regard, BChE fusion protein CocH3-Fc (SEQ ID NO: 9) is more effective in the treatment of organophosphorous treatment than wild-type BChE, which, as noted above, is expected to have the same reaction rate as endogenous BChE. Thus, because the BChE fusion proteins described herein with respect to SEQ ID NO: 10, SEQ ID NO: 11, SEQ ID NO: 12, SEQ ID NO: 13, SEQ ID NO: 14, SEQ ID NO: 15, and SEQ ID NO: 16 all share a similar active site with CocH3-Fc (SEQ ID NO: 9), it is expected that such proteins would also react with OPs, such as paraoxon, in similar fashion as CocH3-Fc (SEQ ID NO: 9), and therefore similarly serve as an effective treatment for OP poisoning.

It will be understood that various details of the presently disclosed subject matter can be changed without departing from the scope of the subject matter disclosed herein. Furthermore, the foregoing description is for the purpose of illustration only, and not for the purpose of limitation.

SEQUENCE LISTING
Wild-type BChE Polypeptide
SEQ ID NO: 1
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESAG
AASVSLHLLS PGSHSLFTRA ILQSGSFNAP WAVTSLYEAR NRTLNLAKLT
GCSRENETEI IKCLRNKDPQ EILLNEAFVV PYGTPLSVNF GPTVDGDFLT
DMPDILLELG QFKKTQILVG VNKDEGTAFL VYGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKVL EMTGNIDEAE WEWKAGFHRW
NNYMMDWKNQ FNDYTSKKES CVGL
Wild-type Fc Polypeptide
SEQ ID NO: 2
AEPKSCDKTH TCPPCPAPEL LGGPSVFLFP PKPKDTLMIS RTPEVTCVVV
DVSHEDPEVK FNWYVDGVEV HNAKTKPREE QYNSTYRVVS VLTVLHQDWL
NGKEYKCKVS NKALPAPIEK TISKAKGQPR EPQVYTLPPS RDELTKNQVS
LTCLVKGFYP SDIAVEWESN GQPENNYKTT PPVLDSDGSF FLYSKLTVDK
SRWQQGNVFS CSVMHEALHN HYTQKSLSLS PGK
CocH3
SEQ ID NO: 3
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTPLGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKVL EMTGNIDEAE WEWKAGFHRW
NNYMMDWKNQ FNDYTSKKES CVGL
CocH3G
SEQ ID NO: 4
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTPLGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWGSFFPKVL EMTGNIDEAE WEWKAGFHRW
NNYMMDWKNQ FNDYTSKKES CVGL
CocH5G
SEQ ID NO: 5
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTALGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWGSFFPKVL EMTGNIDEAE WEWKAGFHRW
NNYMMDWKNQ FNDYTSKKES CVGL
Fc(M6)
SEQ ID NO: 6
VEPKSCDKTH TCPPCPAPEL LGGPSVFLFP PKPKDTLYIT REPEVTCVVV
DVSHEDPEVK FNWYVDGVEV HNAKTKPREE QYNSTYRVVS VLTVLHQDWL
NGKEYKCKVS NKALPAPIEK TISKAKGQPR EPQVYTLPPS REEMTKNQVS
LTCLVKGFYP SDIAVEWESN GQPENNYKTT PPVLDSDGSF FLYSKLTVDK
SRWQQGNVFS CSVMHEALHN HYTQKSLSLS PGK
Fc(M3)
SEQ ID NO: 7
VEPKSCDKTH TCPPCPAPEL LGGPSVFLFP PKPKDTLMIS RTPEVTCVVV
DVSHEDPEVK FNWYVDGVEV HNAKTKPREE QYNSTYRVVS VLTVLHQDWL
NGKEYKCKVS NKALPAPIEK TISKAKGQPR EPQVYTLPPS REEMTKNQVS
LTCLVKGFYP SDIAVEWESN GQPENNYKTT PPVLDSDGSF FLYSKLTVDK
SRWQQGNVFS CSVMHEALHN HYTQKSLSLS PGK
Fc(M4)
SEQ ID NO: 8
VEPKSCDKTH TCPPCPAPEL LGGPSVFLFP PKPKDTLYIS RTPEVTCVVV
DVSHEDPEVK FNWYVDGVEV HNAKTKPREE QYNSTYRVVS VLTVLHQDWL
NGKEYKCKVS NKALPAPIEK TISKAKGQPR EPQVYTLPPS REEMTKNQVS
LTCLVKGFYP SDIAVEWESN GQPENNYKTT PPVLDSDGSF FLYSKLTVDK
SRWQQGNVFS CSVMHEALHN HYTQKSLSLS PGK
CocH3-Fc
SEQ ID NO: 9
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTPLGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKV AEPKSCDKTH TCPPCPAPEL
LGGPSVFLFP
PKPKDTLMIS RTPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS RDELTKNQVS LTCLVKGFYP SDIAVEWESN GQPENNYKTT
PPVLDSDGSF FLYSKLTVDK SRWQQGNVFS CSVMHEALHN HYTQKSLSLS
PGK
CocH3-Fc(M6)
SEQ ID NO: 10
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTPLGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKV VEPKSCDKTH TCPPCPAPEL
LGGPSVFLFP
PKPKDTLYIT REPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
CocH3G-Fc(M3)
SEQ ID NO: 11
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTPLGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKV VEPKSCDKTH TCPPCPAPEL
LGGPSVFLFP
PKPKDTLMIS RTPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
CocH3G-Fc(M4)
SEQ ID NO: 12
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTPLGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKV VEPKSCDKTH TCPPCPAPEL
LGGPSVFLFP
PKPKDTLYIS RTPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
CocH3G-Fc(M6)
SEQ ID NO: 13
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTPLGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWGSFFPKV VEPKSCDKTH TCPPCPAPEL
LGGPSVFLFP
PKPKDTLYIT REPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
CocH5G-Fc(M3)
SEQ ID NO: 14
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTALGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWGSFFPKV VEPKSCDKTH TCPPCPAPEL
LGGPSVFLFP
PKPKDTLMIS RTPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
CocH5G-Fc(M4)
SEQ ID NO: 15
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTALGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWGSFFPKV VEPKSCDKTH TCPPCPAPEL
LGGPSVFLFP
PKPKDTLYIS RTPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
CocH5G-Fc(M6)
SEQ ID NO: 16
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESSG
AASVSLHLLS PGSHSLFTRA ILQSGSANAP WAVTSLYEAR NRTLNLAKLT
GCSRENETEI IKCLRNKDPQ EILLNEAFVV PYGTALGVNF GPTVDGDFLT
DMPDILLELG QFKKTQILVG VNKDEGTWFL VGGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWGSFFPKV VEPKSCDKTH TCPPCPAPEL
LGGPSVFLFP
PKPKDTLYIT REPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
BChE-Fc(M3)
SEQ ID NO: 17
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESAG
AASVSLHLLS PGSHSLFTRA ILQSGSFNAP WAVTSLYEAR NRTLNLAKLT
GCSRENETEI IKCLRNKDPQ EILLNEAFVV PYGTPLSVNF GPTVDGDFLT
DMPDILLELG QFKKTQILVG VNKDEGTAFL VYGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKV VEPKSCDKTH TCPPCPAPEL
LGGPSVFLFP
PKPKDTLMIS RTPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
BChE-Fc(M4)
SEQ ID NO: 18
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESAG
AASVSLHLLS PGSHSLFTRA ILQSGSFNAP WAVTSLYEAR NRTLNLAKLT
GCSRENETEI IKCLRNKDPQ EILLNEAFVV PYGTPLSVNF GPTVDGDFLT
DMPDILLELG QFKKTQILVG VNKDEGTAFL VYGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKV VEPKSCDKTH TCPPCPAPEL
LGGPSVFLFP
PKPKDTLYIS RTPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
BChE-Fc(M6)
SEQ ID NO: 19
EDDIIIATKN GKVRGMNLTV FGGTVTAFLG IPYAQPPLGR LRFKKPQSLT
KWSDIWNATK YANSCCQNID QSFPGFHGSE MWNPNTDLSE DCLYLNVWIP
APKPKNATVL IWIYGGGFQT GTSSLHVYDG KFLARVERVI VVSMNYRVGA
LGFLALPGNP EAPGNMGLFD QQLALQWVQK NIAAFGGNPK SVTLFGESAG
AASVSLHLLS PGSHSLFTRA ILQSGSFNAP WAVTSLYEAR NRTLNLAKLT
GCSRENETEI IKCLRNKDPQ EILLNEAFVV PYGTPLSVNF GPTVDGDFLT
DMPDILLELG QFKKTQILVG VNKDEGTAFL VYGAPGFSKD NNSIITRKEF
QEGLKIFFPG VSEFGKESIL FHYTDWVDDQ RPENYREALG DVVGDYNFIC
PALEFTKKFS EWGNNAFFYY FEHRSSKLPW PEWMGVMHGY EIEFVFGLPL
ERRDNYTKAE EILSRSIVKR WANFAKYGNP NETQNNSTSW PVFKSTEQKY
LTLNTESTRI MTKLRAQQCR FWTSFFPKV VEPKSCDKTH TCPPCPAPEL
LGGPSVFLFP
PKPKDTLYIT REPEVTCVVV DVSHEDPEVK FNWYVDGVEV HNAKTKPREE
QYNSTYRVVS VLTVLHQDWL NGKEYKCKVS NKALPAPIEK TISKAKGQPR
EPQVYTLPPS REEMTKNQVS LTCLVKGFYP SDIAVEWESN GQPENNYKTT
PPVLDSDGSF FLYSKLTVDK SRWQQGNVFS CSVMHEALHN HYTQKSLSLS
PGK
Nucleotide Encoding SEQ ID NO: 1
SEQ ID NO: 20
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
GCA GGA GCA GCT TCA GTT AGC CTG CAT TTG CTT
TCT CCT GGA AGC CAT TCA TTG TTC ACC AGA GCC
ATT CTG CAA AGT GGT TCC TTT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
TCA GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA GCT TTT TTA
GTC TAT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC TTG GAA ATG ACA GGA AAT ATT GAT GAA GCA
GAA TGG GAG TGG AAA GCA GGA TTC CAT CGC TGG
AAC AAT TAC ATG ATG GAC TGG AAA AAT CAA TTT
AAC GAT TAC ACT AGC AAG AAA GAA AGT TGT GTG
GGT CTC
Nucleotide Encoding SEQ ID NO: 2
SEQ ID NO: 21
GCA GAG CCT AAG TCC TGC GAC AAA ACT CAC ACA
TGC CCA CCG TGC CCA GCA CCT GAA CTC CTG GGG
GGA CCG TCA GTC TTC CTC TTC CCC CCA AAA CCC
AAG GAC ACC CTC ATG ATC TCC CGG ACC CCT GAG
GTC ACA TGC GTG GTG GTG GAC GTG AGC CAC GAA
GAC CCT GAG GTC AAG TTC AAC TGG TAC GTG GAC
GGC GTG GAG GTG CAT AAT GCC AAG ACA AAG CCG
CGG GAG GAG CAG TAC AAC AGC ACG TAC CGT GTG
GTC AGC GTC CTC ACC GTC CTG CAC CAG GAC TGG
CTG AAT GGC AAG GAG TAC AAG TGC AAG GTC TCC
AAC AAA GCC CTC CCA GCC CCC ATC GAG AAA ACC
ATC TCC AAA GCC AAA GGG CAG CCC CGA GAA CCA
CAG GTG TAC ACC CTG CCC CCA TCC CGG GAC GAG
CTG ACC AAG AAC CAG GTC AGC CTG ACC TGC CTG
GTC AAA GGC TTC TAT CCC AGC GAC ATC GCC GTG
GAG TGG GAG AGC AAT GGG CAG CCG GAG AAC AAC
TAC AAG ACC ACG CCT CCC GTG CTG GAC TCC GAC
GGC TCC TTC TTC CTC TAC AGC AAG CTC ACC GTG
GAC AAG AGC AGG TGG CAG CAG GGG AAC GTC TTC
TCA TGC TCC GTG ATG CAC GAG GCT CTG CAC AAC
CAC TAC ACG CAG AAG AGC CTC TCC CTG TCT CCG
GGT AAA
Nucleotide Encoding SEQ ID NO: 3
SEQ ID NO: 22
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC TTG GAA ATG ACA GGA AAT ATT GAT GAA GCA
GAA TGG GAG TGG AAA GCA GGA TTC CAT CGC TGG
AAC AAT TAC ATG ATG GAC TGG AAA AAT CAA TTT
AAC GAT TAC ACT AGC AAG AAA GAA AGT TGT GTG
GGT CTC
Nucleotide Encoding SEQ ID NO: 4
SEQ ID NO: 23
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG GCT TCA TTT TTT CCA AAA
GTC TTG GAA ATG ACA GGA AAT ATT GAT GAA GCA
GAA TGG GAG TGG AAA GCA GGA TTC CAT CGC TGG
AAC AAT TAC ATG ATG GAC TGG AAA AAT CAA TTT
AAC GAT TAC ACT AGC AAG AAA GAA AGT TGT GTG
GGT CTC
Nucleotide Encoding SEQ ID NO: 5
SEQ ID NO: 24
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT GCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG GCT TCA TTT TTT CCA AAA
GTC TTG GAA ATG ACA GGA AAT ATT GAT GAA GCA
GAA TGG GAG TGG AAA GCA GGA TTC CAT CGC TGG
AAC AAT TAC ATG ATG GAC TGG AAA AAT CAA TTT
AAC GAT TAC ACT AGC AAG AAA GAA AGT TGT GTG
GGT CTC
Nucleotide Encoding SEQ ID NO: 6
SEQ ID NO: 25
GTG GAG CCT AAG TCC TGC GAC AAA ACT CAC ACA
TGC CCA CCG TGC CCA GCA CCT GAA CTC CTG GGG
GGA CCG TCA GTC TTC CTC TTC CCC CCA AAA CCC
AAG GAC ACC CTC TAT ATC ACC CGG GAA CCT GAG
GTC ACA TGC GTG GTG GTG GAC GTG AGC CAC GAA
GAC CCT GAG GTC AAG TTC AAC TGG TAC GTG GAC
GGC GTG GAG GTG CAT AAT GCC AAG ACA AAG CCG
CGG GAG GAG CAG TAC AAC AGC ACG TAC CGT GTG
GTC AGC GTC CTC ACC GTC CTG CAC CAG GAC TGG
CTG AAT GGC AAG GAG TAC AAG TGC AAG GTC TCC
AAC AAA GCC CTC CCA GCC CCC ATC GAG AAA ACC
ATC TCC AAA GCC AAA GGG CAG CCC CGA GAA CCA
CAG GTG TAC ACC CTG CCC CCA TCC CGG GAG GAG
ATG ACC AAG AAC CAG GTC AGC CTG ACC TGC CTG
GTC AAA GGC TTC TAT CCC AGC GAC ATC GCC GTG
GAG TGG GAG AGC AAT GGG CAG CCG GAG AAC AAC
TAC AAG ACC ACG CCT CCC GTG CTG GAC TCC GAC
GGC TCC TTC TTC CTC TAC AGC AAG CTC ACC GTG
GAC AAG AGC AGG TGG CAG CAG GGG AAC GTC TTC
TCA TGC TCC GTG ATG CAC GAG GCT CTG CAC AAC
CAC TAC ACG CAG AAG AGC CTC TCC CTG TCT CCG
GGT AAA
Nucleotide Encoding SEQ ID NO: 7
SEQ ID NO: 26
GTG GAG CCT AAG TCC TGC GAC AAA ACT CAC ACA
TGC CCA CCG TGC CCA GCA CCT GAA CTC CTG GGG
GGA CCG TCA GTC TTC CTC TTC CCC CCA AAA CCC
AAG GAC ACC CTC ATG ATC TCC CGG ACC CCT GAG
GTC ACA TGC GTG GTG GTG GAC GTG AGC CAC GAA
GAC CCT GAG GTC AAG TTC AAC TGG TAC GTG GAC
GGC GTG GAG GTG CAT AAT GCC AAG ACA AAG CCG
CGG GAG GAG CAG TAC AAC AGC ACG TAC CGT GTG
GTC AGC GTC CTC ACC GTC CTG CAC CAG GAC TGG
CTG AAT GGC AAG GAG TAC AAG TGC AAG GTC TCC
AAC AAA GCC CTC CCA GCC CCC ATC GAG AAA ACC
ATC TCC AAA GCC AAA GGG CAG CCC CGA GAA CCA
CAG GTG TAC ACC CTG CCC CCA TCC CGG GAG GAG
ATG ACC AAG AAC CAG GTC AGC CTG ACC TGC CTG
GTC AAA GGC TTC TAT CCC AGC GAC ATC GCC GTG
GAG TGG GAG AGC AAT GGG CAG CCG GAG AAC AAC
TAC AAG ACC ACG CCT CCC GTG CTG GAC TCC GAC
GGC TCC TTC TTC CTC TAC AGC AAG CTC ACC GTG
GAC AAG AGC AGG TGG CAG CAG GGG AAC GTC TTC
TCA TGC TCC GTG ATG CAC GAG GCT CTG CAC AAC
CAC TAC ACG CAG AAG AGC CTC TCC CTG TCT CCG
GGT AAA
Nucleotide Encoding SEQ ID NO: 8
SEQ ID NO: 27
GTG GAG CCT AAG TCC TGC GAC AAA ACT CAC ACA
TGC CCA CCG TGC CCA GCA CCT GAA CTC CTG GGG
GGA CCG TCA GTC TTC CTC TTC CCC CCA AAA CCC
AAG GAC ACC CTC TAT ATC TCC CGG ACC CCT GAG
GTC ACA TGC GTG GTG GTG GAC GTG AGC CAC GAA
GAC CCT GAG GTC AAG TTC AAC TGG TAC GTG GAC
GGC GTG GAG GTG CAT AAT GCC AAG ACA AAG CCG
CGG GAG GAG CAG TAC AAC AGC ACG TAC CGT GTG
GTC AGC GTC CTC ACC GTC CTG CAC CAG GAC TGG
CTG AAT GGC AAG GAG TAC AAG TGC AAG GTC TCC
AAC AAA GCC CTC CCA GCC CCC ATC GAG AAA ACC
ATC TCC AAA GCC AAA GGG CAG CCC CGA GAA CCA
CAG GTG TAC ACC CTG CCC CCA TCC CGG GAG GAG
ATG ACC AAG AAC CAG GTC AGC CTG ACC TGC CTG
GTC AAA GGC TTC TAT CCC AGC GAC ATC GCC GTG
GAG TGG GAG AGC AAT GGG CAG CCG GAG AAC AAC
TAC AAG ACC ACG CCT CCC GTG CTG GAC TCC GAC
GGC TCC TTC TTC CTC TAC AGC AAG CTC ACC GTG
GAC AAG AGC AGG TGG CAG CAG GGG AAC GTC TTC
TCA TGC TCC GTG ATG CAC GAG GCT CTG CAC AAC
CAC TAC ACG CAG AAG AGC CTC TCC CTG TCT CCG
GGT AAA
Nucleotide Encoding SEQ ID NO: 9
SEQ ID NO: 28
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC GCA GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC ATG ATC TCC CGG ACC CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAC
GAG CTG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 10
SEQ ID NO: 29
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC TAT ATC ACC CGG GAA CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 11
SEQ ID NO: 30
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC ATG ATC TCC CGG ACC CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 12
SEQ ID NO: 31
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC TAT ATC TCC CGG ACC CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 13
SEQ ID NO: 32
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG GCT TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC TAT ATC ACC CGG GAA CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 14
SEQ ID NO: 33
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT GCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG GCT TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC ATG ATC TCC CGG ACC CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 15
SEQ ID NO: 34
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT GCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG GCT TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC TAT ATC TCC CGG ACC CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 16
SEQ ID NO: 35
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
TCC GGA GCA GCT TCA GTT AGC CTG CAT TTG CTT
TCT CCT GGA AGC CAT TCA TTG TTC ACC AGA GCC
ATT CTG CAA AGT GGT TCC GCT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT GCT TTG
GGT GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA TGG TTT TTA
GTC GGT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG GCT TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC TAT ATC ACC CGG GAA CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 17
SEQ ID NO: 36
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
GCA GGA GCA GCT TCA GTT AGC CTG CAT TTG CTT
TCT CCT GGA AGC CAT TCA TTG TTC ACC AGA GCC
ATT CTG CAA AGT GGT TCC TTT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
TCA GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA GCT TTT TTA
GTC TAT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC ATG ATC TCC CGG ACC CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 18
SEQ ID NO: 37
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
GCA GGA GCA GCT TCA GTT AGC CTG CAT TTG CTT
TCT CCT GGA AGC CAT TCA TTG TTC ACC AGA GCC
ATT CTG CAA AGT GGT TCC TTT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
TCA GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA GCT TTT TTA
GTC TAT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC TAT ATC TCC CGG ACC CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA
Nucleotide Encoding SEQ ID NO: 19
SEQ ID NO: 38
GAA GAT GAC ATC ATA ATT GCA ACA AAG AAT GGA
AAA GTC AGA GGG ATG AAC TTG ACA GTT TTT GGT
GGC ACG GTA ACA GCC TTT CTT GGA ATT CCC TAT
GCA CAG CCA CCT CTT GGT AGA CTT CGA TTC AAA
AAG CCA CAG TCT CTG ACC AAG TGG TCT GAT ATT
TGG AAT GCC ACA AAA TAT GCA AAT TCT TGC TGT
CAG AAC ATA GAT CAA AGT TTT CCA GGC TTC CAT
GGA TCA GAG ATG TGG AAC CCA AAC ACT GAC CTC
AGT GAA GAC TGT TTA TAT CTA AAT GTA TGG ATT
CCA GCA CCT AAA CCA AAA AAT GCC ACT GTA TTG
ATA TGG ATT TAT GGT GGT GGT TTT CAA ACT GGA
ACA TCA TCT TTA CAT GTT TAT GAT GGC AAG TTT
CTG GCT CGG GTT GAA AGA GTT ATT GTA GTG TCA
ATG AAC TAT AGG GTG GGT GCC CTA GGA TTC TTA
GCT TTG CCA GGA AAT CCT GAG GCT CCA GGG AAC
ATG GGT TTA TTT GAT CAA CAG TTG GCT CTT CAG
TGG GTT CAA AAA AAT ATA GCA GCC TTT GGT GGA
AAT CCT AAA AGT GTA ACT CTC TTT GGA GAA AGT
GCA GGA GCA GCT TCA GTT AGC CTG CAT TTG CTT
TCT CCT GGA AGC CAT TCA TTG TTC ACC AGA GCC
ATT CTG CAA AGT GGT TCC TTT AAT GCT CCT TGG
GCG GTA ACA TCT CTT TAT GAA GCT AGG AAC AGA
ACG TTG AAC TTA GCT AAA TTG ACT GGT TGC TCT
AGA GAG AAT GAG ACT GAA ATA ATC AAG TGT CTT
AGA AAT AAA GAT CCC CAA GAA ATT CTT CTG AAT
GAA GCA TTT GTT GTC CCC TAT GGG ACT CCT TTG
TCA GTA AAC TTT GGT CCG ACC GTG GAT GGT GAT
TTT CTC ACT GAC ATG CCA GAC ATA TTA CTT GAA
CTT GGA CAA TTT AAA AAA ACC CAG ATT TTG GTG
GGT GTT AAT AAA GAT GAA GGG ACA GCT TTT TTA
GTC TAT GGT GCT CCT GGC TTC AGC AAA GAT AAC
AAT AGT ATC ATA ACT AGA AAA GAA TTT CAG GAA
GGT TTA AAA ATA TTT TTT CCA GGA GTG AGT GAG
TTT GGA AAG GAA TCC ATC CTT TTT CAT TAC ACA
GAC TGG GTA GAT GAT CAG AGA CCT GAA AAC TAC
CGT GAG GCC TTG GGT GAT GTT GTT GGG GAT TAT
AAT TTC ATA TGC CCT GCC TTG GAG TTC ACC AAG
AAG TTC TCA GAA TGG GGA AAT AAT GCC TTT TTC
TAC TAT TTT GAA CAC CGA TCC TCC AAA CTT CCG
TGG CCA GAA TGG ATG GGA GTG ATG CAT GGC TAT
GAA ATT GAA TTT GTC TTT GGT TTA CCT CTG GAA
AGA AGA GAT AAT TAC ACA AAA GCC GAG GAA ATT
TTG AGT AGA TCC ATA GTG AAA CGG TGG GCA AAT
TTT GCA AAA TAT GGG AAT CCA AAT GAG ACT CAG
AAC AAT AGC ACA AGC TGG CCT GTC TTC AAA AGC
ACT GAA CAA AAA TAT CTA ACC TTG AAT ACA GAG
TCA ACA AGA ATA ATG ACG AAA CTA CGT GCT CAA
CAA TGT CGA TTC TGG ACA TCA TTT TTT CCA AAA
GTC GTG GAG CCT AAG TCC TGC GAC AAA ACT CAC
ACA TGC CCA CCG TGC CCA GCA CCT GAA CTC CTG
GGG GGA CCG TCA GTC TTC CTC TTC CCC CCA AAA
CCC AAG GAC ACC CTC TAT ATC ACC CGG GAA CCT
GAG GTC ACA TGC GTG GTG GTG GAC GTG AGC CAC
GAA GAC CCT GAG GTC AAG TTC AAC TGG TAC GTG
GAC GGC GTG GAG GTG CAT AAT GCC AAG ACA AAG
CCG CGG GAG GAG CAG TAC AAC AGC ACG TAC CGT
GTG GTC AGC GTC CTC ACC GTC CTG CAC CAG GAC
TGG CTG AAT GGC AAG GAG TAC AAG TGC AAG GTC
TCC AAC AAA GCC CTC CCA GCC CCC ATC GAG AAA
ACC ATC TCC AAA GCC AAA GGG CAG CCC CGA GAA
CCA CAG GTG TAC ACC CTG CCC CCA TCC CGG GAG
GAG ATG ACC AAG AAC CAG GTC AGC CTG ACC TGC
CTG GTC AAA GGC TTC TAT CCC AGC GAC ATC GCC
GTG GAG TGG GAG AGC AAT GGG CAG CCG GAG AAC
AAC TAC AAG ACC ACG CCT CCC GTG CTG GAC TCC
GAC GGC TCC TTC TTC CTC TAC AGC AAG CTC ACC
GTG GAC AAG AGC AGG TGG CAG CAG GGG AAC GTC
TTC TCA TGC TCC GTG ATG CAC GAG GCT CTG CAC
AAC CAC TAC ACG CAG AAG AGC CTC TCC CTG TCT
CCG GGT AAA

All publications, patents, and patent applications mentioned in this specification are herein incorporated by reference to the same extent as if each individual publication, patent, or patent application was specifically and individually indicated to be incorporated by reference, including the references set forth in the following list:

### REFERENCES

1. Landry, D. W.; Zhao, K.; Yang, G. X.; Glickman, M.; Georgiadis, T. M., Antibody-catalyzed degradation of cocaine. Science 1993, 259 (5103), 1899-1901.
2. Milton, A. L.; Everitt, B. J., Wiping Drug Memories. Science 2012, 336, 167-168.
3. Ersche, K. D.; Jones, P. S.; Williams, G. B.; Turton, A. J.; Robbins, T. W.; Bullmore, E. T., Abnormal Brain Structure Implicated in Stimulant Drug Addiction. Science 2012, 335, 601-604.
4. Gorelick, D. A.; Gardner, E. L.; Xi, Z. X., Agents in development for the management of cocaine abuse. Drugs 2004, 64, 1547-1573.
5. Huang, X.; Gu, H. H.; Zhan, C.-G., Mechanism for cocaine blocking the transport of dopamine: insights from molecular modeling and dynamics simulations. The Journal of Physical Chemistry B 2009, 113 (45), 15057-15066.
6. Yuan, Y.; Huang, X.; Midde, N. M.; Quizon, P. M.; Sun, W. L.; Zhu, J.; Zhan, C.-G., Molecular mechanism of HIV-1 Tat interacting with human dopamine transporter. ACS Chem. Neurosci. 2015, 6, 658-665.
7. Yuan, Y.; Huang, X.; Zhu, J.; Zhan, C.-G., Computational modeling of human dopamine transporter structures, mechanism and its interaction with HIV-1 transactivator of transcription. Future Med. Chem. 2016, 8, 2077-2089.
8. Skolnick, P.; White, D.; Acri, J. B., Editorial: Emerging Targets for Stimulant Use Disorders: Where To Invest In An Era Of Constrained Resources? CNS & Neurological Disorders—Drug Targets 2015, 14, 691.
9. Zheng, F.; Zhan, C.-G., Recent progress in protein drug design and discovery with a focus on novel approaches to the development of anti-cocaine medications. Future Med. Chem. 2009, 1, 515-528.
10. Zheng, F.; Zhan, C.-G., Enzyme therapy approaches for treatment of drug overdose and addiction. Future Med. Chem. 2011, 3, 9-13.
11. Zheng, F.; Zhan, C.-G., Are pharmacokinetic approaches feasible for treatment of cocaine addiction and overdose? Future Med. Chem. 2012, 4, 125-128.
12. Zheng, F.; Zhan, C.-G., Modeling of pharmacokinetics of cocaine in human reveals the feasibility for development of enzyme therapies for drugs of abuse. PLoS Comput. Biol. 2012, 8, e1002610.
13. Hou, S.; Xue, L.; Yang, W.; Fang, L.; Zheng, F.; Zhan, C.-G., Substrate selectivity of high-activity mutants of human butyrylcholinesterase. Org. Biomol. Chem. 2013, 11, 7477-7485.
14. Gorelick, D. A., Pharmacokinetic approaches to treatment of drug addiction. Exp. Rev. Clin. Pharmacol. 2008, 1 (2), 277-290.
15. Zhan, C.-G.; Zheng, F.; Landry, D. W., Fundamental reaction mechanism for cocaine hydrolysis in human butyrylcholinesterase. J. Am. Chem. Soc. 2003, 125, 2462-2474.
16. Sun, H.; Pang, Y.-P.; Lockridge, O.; Brimijoin, S., Re-engineering Butyrylcholinesterase as a Cocaine Hydrolase. Mol. Pharmacol. 2002, 62 (2), 220-224.
17. Pan, Y.; Gao, D.; Yang, W.; Cho, H.; Yang, G.; Tai, H.-H.; Zhan, C.-G., Computational redesign of human butyrylcholinesterase for anticocaine medication. Proc. Natl. Acad. Sci. USA 2005, 102 (46), 16656-16661.
18. Zheng, F.; Yang, W. C.; Ko, M. C.; Liu, J. J.; Cho, H.; Gao, D. Q.; Tong, M.; Tai, H. H.; Woods, J. H.; Zhan, C.-G., Most Efficient Cocaine Hydrolase Designed by Virtual Screening of Transition States. J. Am. Chem. Soc. 2008, 130 (36), 12148-12155.
19. Xue, L.; Ko, M.-C.; Tong, M.; Yang, W.; Hou, S.; Fang, L.; Liu, J.; Zheng, F.; Woods, J. H.; Tai, H.-H.; Zhan, C.-G., Design, preparation, and characterization of high-activity mutants of human butyrylcholinesterase specific for detoxification of cocaine. Mol. Pharmacol. 2011, 79, 290-297.
20. Zheng, F.; Xue, L.; Hou, S.; Liu, J.; Zhan, M.; Yang, W.; Zhan, C.-G., A highly efficient cocaine-detoxifying enzyme obtained by computational design. Nature Commun. 2014, 5, 3457. doi: 10.1388/ncomms4457.
21. Zheng, F.; Yang, W.; Xue, L.; Hou, S.; Liu, J.; Zhan, C.-G., Design of high-activity mutants of human butyrylcholinesterase against (−)-cocaine: structural and energetic factors affecting the catalytic efficiency. Biochemistry 2010, 49, 9113-9119.
22. Zhan, M.; Hou, S.; Zhan, C.-G.; Zheng, F., Kinetic characterization of high-activity mutants of human butyrylcholinesterase for the cocaine metabolite norcocaine. Biochem. J. 2014, 457, 197-206.
23. Hou, S.; Zhan, M.; Zheng, X.; Zhan, C.-G.; Zheng, F., Kinetic characterization of human butyrylcholinesterase mutants for hydrolysis of cocaethylene. Biochem. J. 2014, 460, 447-457.
24. Yang, W.; Xue, L.; Fang, L.; Chen, X.; Zhan, C.-G., Characterization of a high-activity mutant of human butyrylcholinesterase against (−)-cocaine. Chem. Biol. Interact. 2010, 187 (1-3), 148-152.
25. Zhang, T.; Zheng, X.; Zhou, Z.; Chen, X.; Jin, Z.; Deng, J.; Zhan, C.-G.; Zheng, F., Clinical potential of an enzyme-based novel therapy for cocaine overdose. Sci. Rep. 2017, 7, 15303.
26. Zheng, X.; Zhou, Z.; Zhang, T.; Jin, Z.; Chen, X.; Deng, J.; Zhan, C.-G.; Zheng, F., Effectiveness of a cocaine hydrolase for cocaine toxicity treatment in male and female rats. AAPS J. 2018, 20, 3. https://doi.org/10.1208/s12248-017-0167-4.
27. Brimijoin, S.; Gao, Y.; Anker, J. J.; Gliddon, L. A.; LaFleur, D.; Shah, R.; Zhao, Q.; Singh, M.; Carroll, M. E., A Cocaine Hydrolase Engineered from Human Butyrylcholinesterase Selectively Blocks Cocaine Toxicity and Reinstatement of Drug Seeking in Rats. Neumopsychopharmacology 2008, 33 (11), 2715-2725.
28. Cohen-Barak, O.; Wildeman, J.; van de Wetering, J.; Hettinga, J.; Schuilenga-Hut, P.; Gross, A.; Clark, S.; Bassan, M.; Gilgun-Sherki, Y.; Mendzelevski, B.; Spiegelstein, O., Safety, Pharmacokinetics, and Pharmacodynamics of TV-1380, a Novel Mutated Butyrylcholinesterase Treatment for Cocaine Addiction, After Single and Multiple Intramuscular Injections in Healthy Subjects. J. Clin. Pharmacol. 2015, 55, 573-583.
29. Shram, M. J.; Cohen-Barak, O.; Chakraborty, B.; Bassan, M.; Schoedel, K. A.; Hallak, H.; Eyal, E.; Weiss, S.; Gilgun, Y.; Sellers, E. M.; Faulknor, J.; Spiegelstein, O., Assessment of Pharmacokinetic and Pharmacodynamic Interactions Between Albumin-Fused Mutated Butyrylcholinesterase and Intravenously Administered Cocaine in Recreational Cocaine Users. J. Clin. Psychopharmacol. 2015, 35, 396-405.
30. Willyard, C., Quest for the quitting pill. Nature 2015, 522, S53.
31. Gilgun-Sherki, Y.; Eliaz, R. E.; McCann, D. J.; Loupe, P. S.; Eyal, E.; Blatt, K.; Cohen-Barak, O.; Hallak, H.; Chiang, N.; Gyaw, S., Placebo-controlled evaluation of a bioengineered, cocaine-metabolizing fusion protein, TV-1380 (AlbuBChE), in the treatment of cocaine dependence. Drug Alcohol Depend. 2016, 166, 13-20.
32. Shemesh-Darvish, L.; Shinar, D.; Hallak, H.; Gross, A.; Rosenstock, M., TV-1380 attenuates cocaine-induced changes in cardiodynamic parameters in monkeys and reduces the formation of cocaethylene. Drug Alcohol Depend. 2018, 188, 295-303.
33. Schindler, C. W.; Justinova, Z.; Lafleur, D.; Woods, D.; Roschke, V.; Hallak, H.; Sklair-Tavron, L.; Redhi, G. H.; Yasar, S.; Bergman, J.; Goldberg, S. R., Modification of pharmacokinetic and abuse-related effects of cocaine by human-derived cocaine hydrolase in monkeys. Addict. Biol. 2013, 18, 30-39.
34. Zlebnik, N. E.; Brimijoin, S.; Gao, Y.; Saykao, A. T.; Parks, R. J.; Carroll, M. E., Long-term reduction of cocaine self-administration in rats treated with adenoviral vector-delivered cocaine hydrolase: evidence for enzymatic activity. Neuropsychopharmacology 2014, 39, 1538-1546.
35. Li, Y.; Kong, Q.; Yue, J.; Gou, X.; Xu, M.; Wu, X., Genome-edited skin epidermal stem cells protect mice from cocaine-seeking behaviour and cocaine overdose. Nature Biomedical Engineering 2018.
36. Cai, Y.; Zhou, S.; Jin, Z.; Wei, H.; Shang, L.; Deng, J.; Zhan, C.-G.; Zheng, F., Reengineering of Albumin-Fused Cocaine Hydrolase CocH1 (TV-1380) to Prolong Its Biological Half-Life. AAPSJ. 2019, 22, 5. doi: 10.1208/s12248-019-0377-z.
37. Chen, X.; Xue, L.; Hou, S.; Jin, Z.; Zhang, T.; Zheng, F.; Zhan, C.-G., Long-acting cocaine hydrolase for addiction therapy. Proc. Natl. Acad. Sci. USA 2016, 113, 422-427.
38. Chen, X.; Deng, J.; Zheng, X.; Zhang, J.; Zhou, Z.; Wei, H.; Zhan, C.-G.; Zheng, F., Development of a long-acting Fc-fused cocaine hydrolase with improved yield of protein expression. Chem. Biol. Interact. 2019, 306, 89-95.
39. Schrodinger, LLC, The PyMOL Molecular Graphics System, Version 1.8. 2015.
40. Nachon, F.; Carletti, E.; Ronco, C.; Trovaslet, M.; Nicolet, Y.; Jean, L.; Renard, P.-Y., Crystal structures of human cholinesterases in complex with huprine W and tacrine: elements of specificity for anti-Alzheimer's drugs targeting acetyl-and butyryl-cholinesterase. Biochemical Journal 2013, 453 (3), 393-399.
41. Oganesyan, V.; Damschroder, M. M.; Cook, K. E.; Li, Q.; Gao, C.; Wu, H.; Dall'Acqua, W. F., Structural insights into neonatal Fc receptor-based recycling mechanisms. Journal of Biological Chemistry 2014, 289 (11), 7812-7824.
42. Jorgensen, W. L.; Chandrasekhar, J.; Madura, J. D.; Impey, R. W.; Klein, M. L., COMPARISON OF SIMPLE POTENTIAL FUNCTIONS FOR SIMULATING LIQUID WATER. J Chem Phys 1983, 79 (2), 926-935.
43. Case, D. A.; Betz, R. M.; Cerutti, D. S.; T. E. Cheatham, I.; Darden, T. A.; Duke, R. E.; Giese, T. J.; Gohlke, H.; Goetz, A. W.; Homeyer, N.; Izadi, S.; Janowski, P.; Kaus, J.; Kovalenko, A.; Lee, T. S.; LeGrand, S.; Li, P.; Lin, C.; Luchko, T.; Luo, R.; Madej, B.; Mermelstein, D.; Merz, K. M.; Monard, G.; Nguyen, H.; Nguyen, H. T.; Omelyan, I.; Onufriev, A.; Roe, D. R.; Roitberg, A.; Sagui, C.; Simmerling, C. L.; Botello-Smith, W. M.; Swails, J.; Walker, R. C.; Wang, J.; Wolf, R. M.; Wu, X.; Xiao, L.; Kollman, P. A., AMBER 16, University of Caihfornia, San Francisco. 2016.
44. Miller III, B. R.; McGee Jr, T. D.; Swails, J. M.; Homeyer, N.; Gohlke, H.; Roitberg, A. E., MMPBSA. py: An efficient program for end-state free energy calculations. J. Chem. Theory Comput. 2012, 8 (9), 3314-3321.
45. Masson, P.; Xie, W.; Froment, M.-T.; Levitsky, V.; Fortier, P.-L.; Albaret, C.; Lockridge, O., Interaction between the peripheral site residues of human butyrylcholinesterase, D70 and Y332, in binding and hydrolysis of substrates. Biochim. Biophys. Acta 1999, 1433, 281-293.
46. Xue, L.; Hou, S.; Tong, M.; Fang, L.; Chen, X.; Jin, Z.; Tai, H.-H.; Zheng, F.; Zhan, C.-G., Preparation and in vivo characterization of a cocaine hydrolase engineered from human butyrylcholinesterase for metabolizing cocaine. Biochem. J. 2013, 453, 447-454.
47. Feng, Y.; Gong, R.; Dimitrov, D. S., Design, expression and characterization of a soluble single-chain functional human neonatal Fc receptor. Protein Expr Purif 2011, 79 (1), 66-71.
48. Lockridge, O.; Schopfer, L. M.; Winger, G.; Woods, J. H., Large Scale Purification of Butyrylcholinesterase from Human Plasma Suitable for Injection into Monkeys; a Potential New Therapeutic for Protection against Cocaine and Nerve Agent Toxicity. J. Med. Chem. Biol. Radiol. Def 2005, 3, nihms5095.
49. Fan, Y. Y.; Neubert, H., Quantitative Analysis of Human Neonatal Fc Receptor (FcRn) Tissue Expression in Transgenic Mice by Online Peptide Immuno-Affinity LC-HRMS. Anal. Chem. 2016, 88, 4239-4247.
50. Treadwell, S. D.; Robinson, T. G., Cocaine use and stroke. Postgrad. Med. J. 200783, 389-394.
51. Roopenian, D. C.; Akilesh, S., FcRn: the neonatal Fc receptor comes of age. Nat. Rev. Immunol. 2007, 7, 715-725.
52. Yang, B.; Hamza, A.; Wang, Y.; Chen, G.; Zhan, C.-G., Computational determination of binding structures and free energies of phosphodiesterase-2 with benzo[1,4]diazepin-2-one derivatives. J. Phys. Chem. B 2010, 114, 16020-16028.
53. Campino, C.; Szecowka, J.; Michelsen, H.; Seron-Ferre, M., Certain large forms of circulating immunoreactive human growth hormone are in fact immunoglobulins. J. Clin. Endocrinol. Metab. 1990, 70, 601-605.
54. EMA, ORENCIA, INN: abatecept—European Medicines Agency (https://www.ema.europa.eu/en/documents/scientific-discussion/orencia-epar-scientific-discussion_en.pdf).
55. Rigby, M. R.; Harris, K. M.; Pinckney, A.; DiMeglio, L. A.; Rendell, M. S.; Felner, E. I.; Dostou, J. M.; Gitelman, S. E.; Griffin, K. J.; Tsalikian, E.; Gottlieb, P. A.; Greenbaum, C. J.; Sherry, N. A.; Moore, W. V.; Monzavi, R.; Willi, S. M.; Raskin, P.; Keyes-Elstein, L.; Long, S. A.; Kanaparthi, S.; Lim, N.; Phippard, D.; Soppe, C. L.; Fitzgibbon, M. L.; McNamara, J.; Nepom, G. T.; Ehlers, M. R., Alefacept provides sustained clinical and immunological effects in new-onset type 1 diabetes patients. J. Clin. Invest. 2015 125, 3285-3296.
56. O. Lockridge, Review of human butyrylcholinesterase structure, function, genetic variants, history of use in the clinic, and potential therapeutic uses, Pharmacol Ther, 148 (2015) 34-46.
57. D. Cerasoli, E. Griffiths, B. Doctor, A. Saxena, J. Fedorko, N. Greig, Q. Yu, Y. Huang, H. Wilgus, C. Karatzas, (07) In vitro and in vivo characterization of recombinant human butyrylcholinesterase (Protexia™) as a potential nerve agent bioscavenger, Chemico-biological interactions, 157 (2005) 362-365.
58. D. E. Lenz, D. Yeung, J. R. Smith, R. E. Sweeney, L. A. Lumley, D. M. Cerasoli, Stoichiometric and catalytic scavengers as protection against nerve agent toxicity: a mini review, Toxicology, 233 (2007) 31-39.
59. Y. Ashani, S. Shapira, D. Levy, A. D. Wolfe, B. P. Doctor, Butyrylcholinesterase and acetylcholinesterase prophylaxis against soman poisoning in mice, Biochem Pharmacol, 41 (1991) 37-41.
60. R. Brandeis, L. Raveh, J. Grunwald, E. Cohen, Y. Ashani, Prevention of soman-induced cognitive deficits by pretreatment with human butyrylcholinesterase in rats, Pharmacology Biochemistry and Behavior, 46 (1993) 889-896.
61. N. Allon, L. Raveh, E. Gilat, E. Cohen, J. Grunwald, Y. Ashani, Prophylaxis against soman inhalation toxicity in guinea pigs by pretreatment alone with human serum butyryicholinesterase, Toxicol Sci, 43 (1998) 121-128.

1. A method of treating cocaine use disorders in a subject, comprising: administering to the subject an effective amount of a butyrylcholinesterase (BChE) fusion protein to a subject in need thereof, the butyrylcholinesterase fusion protein comprising a BChE protein and a Fc polypeptide mutant with one or more substitutions as compared to SEQ ID NO: 2.

2. The method of claim 1, wherein the Fc polypeptide mutant is selected from the group consisting of SEQ ID NO: 6, SEQ ID NO: 7, and SEQ ID NO: 8.

3. The method of claim 2, wherein the Fc Polypeptide mutant is one of SEQ ID NO: 6 and SEQ ID NO: 8.

4. The method of claim 1, wherein the butyrylcholinesterase fusion protein is a sequence selected from the group consisting of SEQ ID NO: 10, SEQ ID NO: 11, SEQ ID NO: 12, SEQ ID NO: 13 SEQ ID NO: 14, SEQ ID NO: 15, SEQ ID NO: 16, SEQ ID NO: 17, SEQ ID NO: 18, and SEQ ID NO: 19.

5. The method of claim 1, wherein the BChE protein is a BChE mutant with one or more amino acid substitutions as compared to SEQ ID NO: 1.

6. The method of claim 5, wherein the BChE mutant is selected from the group consisting of SEQ ID NO: 3, SEQ ID NO: 4, and SEQ ID NO: 5.

7. The method of claim 5, wherein the butyrylcholinesterase fusion protein is a sequence selected from the group consisting of SEQ ID NO: 10, SEQ ID NO: 11, SEQ ID NO: 12, SEQ ID NO: 13 SEQ ID NO: 14, SEQ ID NO: 15, and SEQ ID NO: 16.

8. The method of claim 1, wherein administration occurs once per week.

9. The method of claim 1, wherein the administration is intravenous or intramuscular.

10. The method of claim 1, wherein the subject is a mammal.

11. The method of claim 10, wherein the subject is a human.

12. The method of claim 1, wherein the effective amount is between about 0.075 mg/kg and about 3 mg/kg.

13. A method of treating organophosphorus poisoning in a subject, comprising: administering to the subject an effective amount of a butyrylcholinesterase (BChE) fusion protein to a subject in need thereof, the butyrylcholinesterase fusion protein comprising a BChE protein and a Fc polypeptide mutant with one or more amino acid substitutions as compared to SEQ ID NO: 2.

14. The method of claim 13, wherein the Fc polypeptide mutant is selected from the group consisting of SEQ ID NO: 6, SEQ ID NO: 7, and SEQ ID NO: 8.

15. The method of claim 14, wherein the Fc Polypeptide mutant is one of SEQ ID NO: 6 and SEQ ID NO: 8.

16. The method of claim 13, wherein the butyrylcholinesterase fusion protein is a sequence selected from the group consisting of SEQ ID NO: 10, SEQ ID NO: 11, SEQ ID NO: 12, SEQ ID NO: 13 SEQ ID NO: 14, SEQ ID NO: 15, SEQ ID NO: 16, SEQ ID NO: 17, SEQ ID NO: 18, and SEQ ID NO: 19.

17. The method of claim 13, wherein the BChE protein is a BChE mutant with one or more amino acid substitutions as compared to SEQ ID NO: 1.

18. The method of claim 17, wherein the BChE mutant is selected from the group consisting of SEQ ID NO: 3, SEQ ID NO: 4, and SEQ ID NO: 5.

19. The method of claim 17, wherein the butyrylcholinesterase fusion protein is a sequence selected from the group consisting of SEQ ID NO: 10, SEQ ID NO: 11, SEQ ID NO: 12, SEQ ID NO: 13 SEQ ID NO: 14, SEQ ID NO: 15, and SEQ ID NO: 16.

20. The method of claim 13, wherein the subject is a mammal.


SEQUENCE LISTING (38 sequences)

SEQ ID NO: 1 - protein, 574 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVLEMTGNIDEAEWEWKAGFHRWNNYMMDWKNQFNDYTSKKESCVGL

SEQ ID NO: 2 - protein, 233 aa, Artificial Sequence
  Recombinant
AEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 233 aa, Artificial Sequence; identical to SEQ ID NO: 2
  Recombinant

SEQ ID NO: 4 - protein, 574 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTPLGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWGSFFPKVLEMTGNIDEAEWEWKAGFHRWNNYMMDWKNQFNDYTSKKESCVGL

SEQ ID NO: 5 - protein, 574 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTALGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWGSFFPKVLEMTGNIDEAEWEWKAGFHRWNNYMMDWKNQFNDYTSKKESCVGL

SEQ ID NO: 6 - protein, 233 aa, Artificial Sequence
  Recombinant
VEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 233 aa, Artificial Sequence
  Recombinant
VEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 233 aa, Artificial Sequence
  Recombinant
VEPKSCDKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTPLGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTPLGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTPLGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTPLGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVVEPKSCDKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTPLGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWGSFFPKVVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTALGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWGSFFPKVVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTALGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWGSFFPKVVEPKSCDKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESSGAASVSLHLLSPGSHSLFTRAILQSGSANAPWAVTSLYEARNRTLNLAKLTGCSRENETEIIKCLRNKDPQEILLNEAFVVPYGTALGVNFGPTVDGDFLTDMPDILLELGQFKKTQILVGVNKDEGTWFLVGGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWGSFFPKVVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVVEPKSCDKTHTCPPCPAPELLGGPSVFLFPPKPKDTLYISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 762 aa, Artificial Sequence
  Recombinant
EDDIIIATKNGKVRGMNLTVFGGTVTAFLGIPYAQPPLGRLRFKKPQSLTKWSDIWNATKYANSCCQNIDQSFPGFHGSEMWNPNTDLSEDCLYLNVWIPAPKPKNATVLIWIYGGGFQTGTSSLHVYDGKFLARVERVIVVSMNYRVGALGFLALPGNPEAPGNMGLFDQQLALQWVQKNIAAFGGNPKSVTLFGESAGAASVSLHLLSPGSHSLFTRAILQSGSFNAPWAVTSLYEARNRTLNLAKLTGCSRENETEIIKCLRNKDPQEILLNEAFVVPYGTPLSVNFGPTVDGDFLTDMPDILLELGQFKKTQILVGVNKDEGTAFLVYGAPGFSKDNNSIITRKEFQEGLKIFFPGVSEFGKESILFHYTDWVDDQRPENYREALGDVVGDYNFICPALEFTKKFSEWGNNAFFYYFEHRSSKLPWPEWMGVMHGYEIEFVFGLPLERRDNYTKAEEILSRSIVKRWANFAKYGNPNETQNNSTSWPVFKSTEQKYLTLNTESTRIMTKLRAQQCRFWTSFFPKVVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 1722 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggttcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctc

SEQ ID NO: 21 - DNA, 699 bp, Artificial Sequence
  Recombinant
gcagagcctaagtcctgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 22 - DNA, 1722 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctc

SEQ ID NO: 23 - DNA, 1722 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctgggcttcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctc

SEQ ID NO: 24 - DNA, 1722 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactgctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctgggcttcattttttccaaaagtcttggaaatgacaggaaatattgatgaagcagaatgggagtggaaagcaggattccatcgctggaacaattacatgatggactggaaaaatcaatttaacgattacactagcaagaaagaaagttgtgtgggtctc

SEQ ID NO: 25 - DNA, 699 bp, Artificial Sequence
  Recombinant
gtggagcctaagtcctgcgacaaaactcacacatgcccaccgtgcccagcacctgaactcctggggggaccgtcagtcttcctcttccccccaaaacccaaggacaccctctatatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 26 - DNA, 699 bp, Artificial Sequence
  Recombinant
gtggagcctaagtcctgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 27 - DNA, 699 bp, Artificial Sequence
  Recombinant
gtggagcctaagtcctgcgacaaaactcacacatgcccaccgtgcccagcacctgaactcctggggggaccgtcagtcttcctcttccccccaaaacccaaggacaccctctatatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 28 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcgcagagcctaagtcctgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 29 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcgtggagcctaagtcctgcgacaaaactcacacatgcccaccgtgcccagcacctgaactcctggggggaccgtcagtcttcctcttccccccaaaacccaaggacaccctctatatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 30 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcgtggagcctaagtcctgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 31 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcgtggagcctaagtcctgcgacaaaactcacacatgcccaccgtgcccagcacctgaactcctggggggaccgtcagtcttcctcttccccccaaaacccaaggacaccctctatatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 32 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctgggcttcattttttccaaaagtcgtggagcctaagtcctgcgacaaaactcacacatgcccaccgtgcccagcacctgaactcctggggggaccgtcagtcttcctcttccccccaaaacccaaggacaccctctatatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 33 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactgctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctgggcttcattttttccaaaagtcgtggagcctaagtcctgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 34 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactgctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctgggcttcattttttccaaaagtcgtggagcctaagtcctgcgacaaaactcacacatgcccaccgtgcccagcacctgaactcctggggggaccgtcagtcttcctcttccccccaaaacccaaggacaccctctatatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 35 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagttccggagcagcttcagttagcctgcatttgctttctcctggaagccattcattgttcaccagagccattctgcaaagtggttccgctaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactgctttgggtgtaaactttggtccgaccgtggatggtgattttctcactgacatgccagacatattacttgaacttggacaatttaaaaaaacccagattttggtgggtgttaataaagatgaagggacatggtttttagtcggtggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctgggcttcattttttccaaaagtcgtggagcctaagtcctgcgacaaaactcacacatgcccaccgtgcccagcacctgaactcctggggggaccgtcagtcttcctcttccccccaaaacccaaggacaccctctatatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 36 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggttcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcgtggagcctaagtcctgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 37 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggttcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcgtggagcctaagtcctgcgacaaaactcacacatgcccaccgtgcccagcacctgaactcctggggggaccgtcagtcttcctcttccccccaaaacccaaggacaccctctatatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa

SEQ ID NO: 38 - DNA, 2286 bp, Artificial Sequence
  Recombinant
gaagatgacatcataattgcaacaaagaatggaaaagtcagagggatgaacttgacagtttttggtggcacggtaacagcctttcttggaattccctatgcacagccacctcttggtagacttcgattcaaaaagccacagtctctgaccaagtggtctgatatttggaatgccacaaaatatgcaaattcttgctgtcagaacatagatcaaagttttccaggcttccatggatcagagatgtggaacccaaacactgacctcagtgaagactgtttatatctaaatgtatggattccagcacctaaaccaaaaaatgccactgtattgatatggatttatggtggtggttttcaaactggaacatcatctttacatgtttatgatggcaagtttctggctcgggttgaaagagttattgtagtgtcaatgaactatagggtgggtgccctaggattcttagctttgccaggaaatcctgaggctccagggaacatgggtttatttgatcaacagttggctcttcagtgggttcaaaaaaatatagcagcctttggtggaaatcctaaaagtgtaactctctttggagaaagtgcaggagcagcttcagttagcctgcatttgctttctcctggaagccattcattgttcaccagagccattctgcaaagtggttcctttaatgctccttgggcggtaacatctctttatgaagctaggaacagaacgttgaacttagctaaattgactggttgctctagagagaatgagactgaaataatcaagtgtcttagaaataaagatccccaagaaattcttctgaatgaagcatttgttgtcccctatgggactcctttgtcagtaaactttggtccgaccgtggatggtgattttctcactgacatgccagacatattacttgaacttggacaatttaaaaaaacccagattttggtgggtgttaataaagatgaagggacagcttttttagtctatggtgctcctggcttcagcaaagataacaatagtatcataactagaaaagaatttcaggaaggtttaaaaatattttttccaggagtgagtgagtttggaaaggaatccatcctttttcattacacagactgggtagatgatcagagacctgaaaactaccgtgaggccttgggtgatgttgttggggattataatttcatatgccctgccttggagttcaccaagaagttctcagaatggggaaataatgcctttttctactattttgaacaccgatcctccaaacttccgtggccagaatggatgggagtgatgcatggctatgaaattgaatttgtctttggtttacctctggaaagaagagataattacacaaaagccgaggaaattttgagtagatccatagtgaaacggtgggcaaattttgcaaaatatgggaatccaaatgagactcagaacaatagcacaagctggcctgtcttcaaaagcactgaacaaaaatatctaaccttgaatacagagtcaacaagaataatgacgaaactacgtgctcaacaatgtcgattctggacatcattttttccaaaagtcgtggagcctaagtcctgcgacaaaactcacacatgcccaccgtgcccagcacctgaactcctggggggaccgtcagtcttcctcttccccccaaaacccaaggacaccctctatatcacccgggaacctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaa